(19) United States
(12) Patent Application Publication
(10) Pub. No.: US 2005/0233364 A1
(43) Pub. Date: Oct. 20, 2005

(54) Rapid integration site mapping

(75) Inventors: Shawn Burgess, Bethesda, MD (US); Xiaolin Wu, Gaithersburg, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Government of the U.S.A. as represented by the Secretary of the Dept. of Health & Human Services

(21) Appl. No.: 11/101,299

(22) Filed: Apr. 6, 2005

Related U.S. Application Data
(60) Provisional application No. 60/564,095, filed on Apr. 20, 2004.

(30) Foreign Application Priority Data
Apr. 20, 2004 (CA) 2,465,396

(51) Int. Cl. C12Q 1/68

(52) U.S. Cl. 435/6

(57) ABSTRACT

High-throughput methods for mapping integration sites resulting from one or more integrations, such as infection by a retrovirus, are disclosed. The disclosed methods require no selection for specific phenotypes such as antibiotic resistance, and thereby may avoid selection bias. Moreover, the linker-based amplification is simple and rapid, and by using a frequently cutting restriction enzyme, the amplicons are small, which significantly decreases possible amplification and cloning biases.

### CROSS REFERENCE TO RELATED APPLICATION

This application claims the benefit of U.S. Provisional Application No. 60/564,095, filed Apr. 20, 2004, which is incorporated by reference herein in its entirety.

### FIELD

This disclosure relates to methods of rapidly mapping where integrants have integrated into a nucleic acid molecule, for example, methods of rapidly mapping retroviral integration sites in genomic DNA, and applications of such method.

### BACKGROUND

Retroviruses have been used as an efficient gene delivery vehicle in many gene therapy trials. Historically, retroviral integrations were believed to be random and the chance of accidentally disrupting or activating a gene was considered remote. Recently, two of eleven children treated for a rare blood disease with an MLV-based gene therapy vector developed leukemia, at least in part by insertion of the MLV provirus near the same growth-promoting gene, LMO2 (Check, Nature, 420:116-118, 2002; Kaiser, Science, 299:495, 2003). Thus, the safety of these treatments has become a primary consideration and casts serious doubt on the assumption of random integration.

Although in vitro integration models have identified several factors relating to integration site selection, such as nucleosomal structure and DNA binding proteins (Pryciak and Varmus, Cell, 69:769-780, 1992; Pryciak et al., Proc. Natl. Acad. Sci. USA, 89:9237-9241, 1992; Pryciak et al., EMBO J., 11:291-303, 1992; Pruss et al., J. Biol. Chem., 269:25031-25041, 1994; Pruss et al., Proc. Natl. Acad. Sci. USA, 91:5913-5917, 1994; Bushman, Proc. Natl. Acad. Sci. USA, 91:9233-9237, 1994), integration site selection in vivo still remains poorly understood and no consensus sequences have been determined in the primary flanking sequences of target site DNA. Before the sequence of the human genome was available, it was impossible to obtain an accurate global picture of retroviral integration events. Early in vivo studies have produced conflicting results, with some reporting that transcriptionally active regions are favored for retroviral integration (Scherdin et al., J. Virol., 64:907-912, 1990; Mooslehner et al., J. Virol., 64:3056-3058, 1990), and others reported that transcriptionally active regions are disfavored (Weidhaas et al., J. Virol., 74:8382-8389, 2000). Recently, Schroder et al. mapped over 500 integrations of HIV-1 in the human genome and reported that HIV-1 integration favored genes (Schroder et al., Cell, 110:521-529, 2002).

It will be important to continue to map viral integration sites, for example, to determine whether other virus have specific integration preferences, and to identify viral gene therapy vectors that have safe integration profiles. Unfortunately, methods for mapping viral integration sites, such as described by Schroder et al. (Cell, 110:521-529, 2002), are laborious and time consuming. Several months may be required to map the substantial number of viral integration sites that are necessary to obtain an accurate integration profile. Moreover, existing methods are subject to various biases, such as selection bias, amplification bias and/or cloning bias, each of which may result in an incomplete or inaccurate integration profile. Thus, new, faster, more reliable methods of mapping viral integration sites are needed.

### SUMMARY OF THE DISCLOSURE

High-throughput methods have been developed to identify sites where integrants have integrated into a nucleic acid molecule. Particular methods are described whereby genomic DNA sequences flanking integration sites can be identified. The disclosed methods require no selection for phenotype, such as antibiotic resistance, which might bias the sample. Moreover, the linker-based amplification is simple and rapid, and by using a frequently cutting restriction enzyme (such as, MseI, RsaI, TaqI, Tri1I or RsaI), the resultant amplicons are relatively small, which significantly decreases possible amplification and cloning biases.

With the disclosed methods, it is now feasible to rapidly map integration sites resulting from a particular integration event, such as infection by a retrovirus. Hence, it is now possible to identify the integration profiles for various integrants, including, for example, retroviruses or integrating gene therapy vectors. In some examples, integrating gene therapy vectors may be screened for random or nearer-to-random integration profiles, which are believed to be safer when the vector is administered to patients. In other examples, it is now possible to screen cells that have been treated with an integrating gene therapy vector, for instance, prior to or after administration of such cells to patients. In this way, it is possible to identify vector integrations that may increase the risk of the patient for developing unwanted side effects, such as cancer. Under such circumstances, medical personnel may elect, as applicable, not to administer the infected cells and/or to counsel the patient accordingly. For example, using the disclosed methods, it is now possible to identify insertion of an MLV provirus near the growth-promoting gene, LMO2, in a matter of days.

The foregoing and other features and advantages will become more apparent from the following detailed description of several embodiments, which proceeds with reference to the accompanying figures.

### BRIEF DESCRIPTION OF THE FIGURES

FIG. 1 is a schematic representation of one method embodiment. In this embodiment, amplification of an integration junction fragment containing nucleic acid sequences flanking the 3′ end of a single integrant is illustrated.

FIG. 2 is a diagram of an exemplar integrant.

FIG. 3 is a schematic representation of certain nucleic acid fragments that may be produced by a restriction enzyme digestion step of some method embodiments. Such fragments are not typically amplified in the disclosed methods.

FIG. 4 shows in greater detail the amplification reactions contained within the dashed box of FIG. 1.

FIG. 5 is a diagram comparing the expected outcomes of amplification reactions with and without digestion of the amplification template with N2.

FIG. 6A shows a graph of the distribution of MLV integrations with respect to distance from the transcriptional start site of all RefSeq genes. Windows of varying sizes from 1 kb to 10 kb were selected upstream and downstream of the transcriptional start site for all RefSeq genes. The total numbers of MLV integrations in each window were counted and an average integration rate/kb was calculated. The dashed line represents the expected number of random integrations/kb. FIG. 6B shows a graph of the percentage of the total integrations for MLV and HIV-1 in three separate regions of the RefSeq transcripts: 5 kb upstream, the transcript itself (each transcript is divided into eight equal sections regardless of length), and 5 kb downstream.

FIG. 7 shows a histogram of median expression levels of 1000 sets of 79 random genes on the GSM2145 chip. The median level of genes having an MLV integration within ±5 kb of a transcriptional start is statistically different from a random data set.

FIG. 8 shows a digital representation of a 2% agarose gel used to separate (i) 3′ integration junction fragments amplified from pGT plasmid DNA (lane 1) and isolated GT186 genomic DNA (lane 3), in each case digested with MseI and PstI; and (ii) 5′ integration junction fragments amplified from pGT plasmid DNA (lane 2) and isolated GT186 genomic DNA (lane 4), in each case digested with MseI and EcoRI. Lanes M show molecular weight markers from 100-1000 base pairs in 100 base pair increments. These results, as well as results shown in FIGS. 9 and 10 (below), demonstrate that both 3′ and 5′ integration junction fragments can be obtained using the disclosed methods.

FIG. 9 shows a digital representation of a 2% agarose gel used to separate 3′ and 5′ integration junction fragments amplified from isolated GT186 genomic DNA. To obtain 3′ integration junction fragments, GT186 genomic DNA was digested with MseI and PstI. To obtain 5′ integration junction fragments, GT186 genomic DNA was digested with MseI and EcoRI. The amount of GT186 genomic DNA used in each experiment (250 ng, 50 ng, or 5 ng) is indicated above the respective lanes. These results demonstrate that integration site junctions can be efficiently amplified from no more than 5 ng genomic DNA.

FIG. 10 shows a digital representation of a 2% agarose gel used to separate (i) 5′ integration junction fragments amplified from pGT plasmid DNA (lane 1) and isolated GT186 genomic DNA (lane 3), in each case digested with RsaI and PstI; and (ii) 3′ integration junction fragments amplified from pGT plasmid DNA (lane 2) and isolated GT186 genomic DNA (lane 4), in each case digested with RsaI and EcoRI. Lanes M show molecular weight markers from 100-1000 base pairs in 100 base pair increments. These results demonstrate that various restriction enzymes may be useful as the first restriction enzyme (N1) in the disclosed methods.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 shows a plus strand of an MseI-compatible linker useful in some embodiments of the disclosed methods.

SEQ ID NO: 2 shows a minus strand of an MseI-compatible linker useful in some embodiments of the disclosed methods.

SEQ ID NO: 3 shows an MseI-compatible linker primer useful in some embodiments of the disclosed methods.

SEQ ID NO: 4 shows an MseI-compatible linker nested primer useful in some embodiments of the disclosed methods.

SEQ ID NO: 5 shows a MLV 3′ LTR primer useful in some embodiments of the disclosed methods.

SEQ ID NO: 6 shows a MLV 3′ LTR nested primer useful in some embodiments of the disclosed methods.

SEQ ID NO: 7 shows a HIV-1 3′ LTR primer useful in some embodiments of the disclosed methods.

SEQ ID NO: 8 shows a HIV-1 3′ LTR nested primer useful in some embodiments of the disclosed methods.

SEQ ID NO: 9 shows a plus strand of a RsaI-compatible linker useful in some embodiments of the disclosed methods.

SEQ ID NO: 10 shows a minus strand of a RsaI-compatible linker useful in some embodiments of the disclosed methods.

SEQ ID NO: 11 shows a RsaI-compatible linker primer useful in some embodiments of the disclosed methods.

SEQ ID NO: 12 shows a RsaI-compatible linker nested primer useful in some embodiments of the disclosed methods.

SEQ ID NO: 13 shows a MLV 5′ LTR primer useful in some embodiments of the disclosed methods.

SEQ ID NO: 14 shows a MLV 5′ LTR nested primer useful in some embodiments of the disclosed methods.

### DETAILED DESCRIPTION

I. Overview

Disclosed herein are methods of identifying an integrant integration site, involving steps (a)-(g). Step (a) involves obtaining a nucleic acid molecule including at least one integrant at an integration site and at least one first restriction site (N1 site) cleavable by a first restriction enzyme (N1), wherein the integrant includes in the following order (i) a first terminal repeat, including a target end and a terminal repeat-specific primer (TRP) binding site, which can stably bind a TRP, (ii) at least one second restriction site (N2 site) cleavable by a second restriction enzyme (N2), and (iii) a second terminal repeat, including a non-target end and a sequence, which can stably bind a TRP, and which is in the same orientation as the TRP binding site in the first terminal repeat. Additional steps of disclosed methods involve: (b) digesting the nucleic acid molecule with N1 and N2 to yield a population of nucleic acid fragments, wherein at least some of the fragments have at least one N1 end; (c) ligating an extension-dependent linker to at least some of the N1 ends to produce a population of linkered fragments; (d) contacting the linkered fragments with the TRP; (e) extending the TRP to yield at least one extension product having a linker-specific primer (LSP) binding site complementary to a LSP; (f) amplifying the linkered fragments and extension product(s) with TRPs and LSPs to yield at least one amplification product; and (g) sequencing at least one amplification product to yield at least one nucleic acid sequence flanking the target end, thereby identifying at least one integrant integration site.

In some embodiments, the integrant is a virus, a transposon, or an integrating gene therapy vector and, in particular embodiments, the integrant is a virus, such as murine leukemia virus (MLV) or human immunodeficiency virus 1 (HIV-1). In particular embodiments, the target end is the 3′ end of the integrant, or the target end is the 5′ end of the integrant. In other particular embodiments, the TRP binding site is no more than about 200 base pairs from the target end.

In some method embodiments, the nucleic acid molecule is genomic DNA or, more particularly, is human genomic DNA. In still other embodiments, N1, which digests the nucleic acid molecule, is no more than a 5-base cutter, or is no more than a 4-base cutter. In specific embodiments, N1 is MseI, RsaI, TaqI, Tri1I or RsaI. In some examples, N2 cuts the nucleic acid molecule less frequently than does N1. In another example, N2 is PstI or EcoRI. In some examples, the nucleic acid molecule is co-digested with N1 and N2. In other example, the nucleic acid molecule is sequentially digested with N1 and N2; for example, the nucleic acid molecule is first digested with N1 and then digested with N2. In some embodiments, N1 and N2 produce incompatible ends, while in other embodiments N1 and N2 produce compatible ends.

Certain of the disclosed methods involve a population of nucleic acid fragments having an average length of no more than about 300 base pairs. More particular examples involve an average fragment length of no more than about 100 base pairs.

Some disclosed methods are performed in no more than 14 days, while other disclosed methods are performed in no more than 7 days. In some methods, at least 200 integration sites are identified, and in other methods at least 500 integration sites are identified.

Also disclosed herein are methods of determining the risk potential of an integrating gene therapy vector, involving isolating a nucleic acid molecule, which includes at least one integrated integrating gene therapy vector and at least one reference point, from a treated cell; identifying integration sites of the gene therapy vector according to methods of identifying an integrant integration site described herein; and mapping integration sites in relation to at least one reference point; wherein the map of integration sites provides information about the risk potential of the integrating gene therapy vector.

In some examples, the treated cells include mammalian cells or, in more particular examples, human cells. In some examples, human cells are isolated from a subject to whom the treated cells are to be administered. In other examples, the human cells are isolated from a subject to whom the treated cells were administered.

Some methods involve a nucleic acid molecule, which includes genomic DNA. In other methods, the integrating gene therapy vector includes all or part of the genome from MLV or HIV-1. Still other methods involve a reference point, which includes actively transcribed regions of the nucleic acid molecule or telomeres. In methods involving actively transcribed regions, such regions include translation start sites, transcription start sites, midpoints of coding regions, or stop codons.

In some examples, the risk potential of the integrating gene therapy vector is relatively high when substantial numbers of integration sites are located near actively transcribed regions of the nucleic acid molecule. In other methods, the risk potential of the integrating gene therapy vector is relatively low when the distribution of integration sites is substantially random in relation to actively transcribed regions of the nucleic acid molecule.

In still other methods, substantially all integration sites are mapped.

II. Abbreviations and Terms

HIV-1 human immunodeficiency virus 1
LM-PCR linker-mediated PCR
LSP linker-specific primer
LTR long terminal repeat
MLV murine leukocyte virus
N1 first restriction enzyme
N1 site recognition site of N1
N2 second restriction enzyme
N2 site recognition site of N2
NCBI National Center for Biotechnology Information
PCR polymerase chain reaction
TRP terminal-repeat-specific primer
VSV-G vesicular stomatitis virus glycoprotein G

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a polynucleotide is referred to as the “5′ end” when its 5′ phosphate is not linked to the 3′oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Amplifying a nucleic acid: To increase the number of copies of a nucleic acid. The resulting amplification products are called “amplicons.”

Binding or stable binding: An oligonucleotide (such as, a primer) binds or stably binds to a target nucleic acid if a sufficient amount of the oligonucleotide forms base pairs or is hybridized to its target nucleic acid, to permit detection of that binding. Binding can be detected by either physical or functional properties of the target:oligonucleotide complex. Binding between a target and an oligonucleotide can be detected by any procedure known to one skilled in the art, including both functional and physical binding assays. Binding may be detected functionally by determining whether binding has an observable effect upon a biosynthetic process such as expression of a coding sequence, DNA replication, transcription, amplification and the like. For example, stable binding of a primer (such as a TRP) to a primer binding site (such as a TRP binding site) may be detected by the formation of a primer extension product.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, one method that is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt.

The binding between an oligomer and its target nucleic acid is frequently characterized by the temperature (Tₘ) (under defined ionic strength and pH) at which 50% of the target sequence remains hybridized to a perfectly matched probe or complementary strand. A higher (Tₘ) means a stronger or more stable complex relative to a complex with a lower (Tₘ).

Extension product: A nucleic acid strand produced by extension of an oligonucleotide, such as a primer, via incorporation of deoxynucleotide triphosphates or ribonucleotide triphosphates as mediated by an enzymatic reaction (involving, for example, DNA polymerase) in combination with a template nucleic acid strand. The nucleic acid sequence of an extension product is substantially the complement of the nucleic acid sequence of the template used to synthesize the extension product.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript. “RefSeq genes” are those genes identified in the National Center for Biotechnology Information RefSeq database, which is a curated, non-redundant set of reference sequences including genomic DNA contigs, mRNAs and proteins for known genes, and entire chromosomes (The NCBI handbook [Internet], Bethesda (MD): National Library of Medicine (US), National Center for Biotechnology Information; 2002 Oct. Chapter 18, The Reference Sequence (RefSeq) Project; available from the NCBI website).

Flanking: Near or next to, also, including adjoining, for instance in a linear polynucleotide, such as a DNA molecule. Nucleotides of a nucleic acid molecule that flank an integrant either upstream of the integrant's 5′ end or downstream of the integrant's 3′ end may be more distinctly referred to as “non-integrant flanking sequence(s)”. Non-integrant flanking sequences may include two or more contiguous non-integrant nucleotides. For example, non-integrant flanking sequences may be about 10, about 20, about 30, about 40, about 50, about 75, about 100, or about 250 contiguous base pairs in length. Often, non-integrant flanking sequences may adjoin an integrant sequence. In other examples, non-integrant flanking sequences are not necessarily adjoining an integrant sequence, but are near to the integrant sequence. In particular examples, non-integrant flanking sequences may begin about 5, about 10, about 20, or about 50 base pairs upstream or downstream of the 5′ or 3′ end, respectively, of an integrant.

Gene therapy: The introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects a function of the recipient cell. In another example, the heterologous nucleic acid molecule may encode an anti-sense nucleic acid that is complementary to a nucleic acid molecule present in the recipient cell, and thereby affect a function of the corresponding native nucleic acid molecule. In still other examples, the heterologous nucleic acid may encode a ribozyme or deoxyribozyme, which are capable of cleaving nucleic acid molecules present in the recipient cell. In another example, the heterologous nucleic acid may encode a so-called decoy molecule, which is capable of specifically binding a peptide molecule present in the recipient cell.

Introduction of heterologous nucleic acids into one or more recipient cells is achieved by various methods known in the art. Of particular interest to the disclosed methods are gene delivery vehicles, referred to herein as “integrating gene therapy vectors,” which cause a heterologous nucleic acid molecule, typically together with at least some nucleic acid sequences of the vector, to be integrated into the recipient cell's genomic DNA. In some examples, an integrating gene therapy vector is derived from a virus, including but not limited to adenoviruses, retroviruses, vaccinia viruses or adeno-associated viruses.

Genomic DNA: The DNA originating within the nucleus and containing an organism's genome, which is passed on to its offspring as information for continued replication and/or propagation and/or survival of the organism. The term can be used to distinguish between other types of DNA, such as DNA found within plasmids or organelles. The “genome” is all the genetic material in the chromosomes of a particular organism.

Human Immunodeficiency Virus (HIV): A retrovirus that causes immunosuppression in humans and leads to a disease complex known as acquired immunodeficiency syndrome (AIDS). HIV subtypes can be identified by particular number, such as HIV-1 and HIV-2. More detailed information about HIV can be found in Coffin et al., Retroviruses, Cold Spring Harbor Laboratory Press, 1997.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between to distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

For present purposes, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Representative conditions of hybridization are shown below:

Very High Stringency

| | | | | | | |
|---|---|---|---|---|---|---|
| Hybridization | in | 5x | SSC | at | 65° C. | 16 hours |
| Wash twice | in | 2x | SSC | at | 55° C. | 15 minutes each |
| Wash twice | in | 2x | SSC | at | room temp. | 20 minutes each |

Medium Stringency

| | | | | | | |
|---|---|---|---|---|---|---|
| Hybridization | in | 5x | SSC | at | 42° C. | 16 hours |
| Wash twice | in | 2x | SSC | at | room temp. | 20 minutes each |
| Wash once | in | 2x | SSC | at | 42° C. | 30 minutes each |

Moderate Stringency

| | | | | | | |
|---|---|---|---|---|---|---|
| Hybridization | in | 6x | SSC | at | room temp. | 16 hours |
| Wash twice | in | 2x | SSC | at | room temp. | 20 minutes each |

In vitro amplification: Any one of many techniques used to increase the number of copies of a nucleic acid molecule in a sample or specimen in vitro. An example of in vitro amplification is the polymerase chain reaction (PCR), in which a biological sample collected from a subject is contacted with a pair of oligonucleotide primers, under conditions that allow for the hybridization of the primers to nucleic acid template in the sample. The primers are extended under suitable conditions (to produce an extension product), dissociated from the template, and then re-annealed, extended, and dissociated to amplify the number of copies of the nucleic acid. The product of in vitro amplification (which may be referred to, for example, as an amplicon or an amplification product) may be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Integrant: A nucleic acid molecule that can be (or is) integrated into a nucleic acid molecule. Typically, an integrant will have terminal repeats usually in the same orientation. Integrants include, without limitation, integrating viruses (such as, adenoviruses, retroviruses, vaccinia viruses and adeno-associated viruses), retrotransposons, integrating gene therapy vectors, and other transposable elements (such as, P elements in Drosophila melanogaster and T DNA in various plants). A “retrovirus” is an RNA virus that replicates by first being converted into double-stranded DNA by reverse transcriptase. Representative retroviruses include, without limitation, HIV-1, MLV, murine sarcoma virus (MSV), avian leukosis virus (ALV), human foamy virus (HFV), human T-cell leukemia virus (HTLV-I(II)), and Rous sarcoma virus (RSV). A “transposon” is a transposable DNA element that uses an integrase enzyme to integrate into a target nucleic acid without going through an RNA intermediate. Examples of transposons include, for example, SB (sleeping beauty) P elements, and TOL2 (a transposon isolated from the genome of the medaka fish), and the Ac element (isolated from maize genome). A “retrotransposon” is a transposable DNA element (transposon) that is replicated through an RNA intermediate via reverse transcriptase. Examples include, for example, yeast Ty elements, Drosophila copia elements, and human LINE1 elements.

Integration: The process by which an integrant (such as, an integrating virus, a retrotransposon, an integrating gene therapy vector; or a transposon) becomes incorporated or inserted (“integrated”) into a nucleic acid molecule, for instance into the genomic DNA of one or more target cells. Each location in a nucleic acid molecule into which an integrant is inserted is called an “integration site.”

An “integration junction fragment” refers to a relatively short nucleic acid molecule that contains at least one series of nucleotides that transitions from integrant nucleic acid sequence to non-integrant nucleic acid sequences (also called, an integration site junction), and includes parts of both the integrant and non-integrant nucleic acid. For each integration event, there will typically be a 5′ integration site junction, which is the transition from the 5′ integrant sequence to the upstream non-integrant sequence, and a 3′ integration site junction, which is the transition from the 3′ integrant sequence to the downstream non-integrant sequence. Using the methods disclosed herein, the 5′ integration site junction and the 3′ integration site junction will generally be located on separate integration junction fragments.

A representative integration junction fragment will typically be no more than about 50, 70, 100, 250, 500, or 1000 base pairs in length. The number of nucleotides of an integration junction fragment attributable to an integrant or the target molecule may vary, as long as the integration junction fragment contains at least about 10, at least about 15, at least about 18, at least about 20, at least about 30, or at least about 40 base pairs of non-integrant flanking sequence.

For each integrant, there is a 5′ integration site junction (including 5′ flanking target molecule sequences and at least the 5′ end of an integrant) and a 3′ integration site junction (including 3′ flanking target molecule sequences and at least the 3′ end of an integrant).

Integration profile: The distribution of integrant integration sites with respect to one or more particular reference points, for example, with respect to the distance of the integration from the transcriptional start site of selected populations of genes, such as some or all RefSeq genes, or with respect to the coding regions of selected populations of genes, such as some or all RefSeq genes. An integration profile may also be referred to as a pattern of integration. A particular integrant may have a characteristic integration profile, which may differ from the integration profile of a different integrant.

Ligation: The process of forming phosphodiester bonds between two or more polynucleotides, such as between double-stranded DNAs, or between a linker and an integration junction fragment. Techniques for ligation are well known to the art and protocols for ligation are described in standard laboratory manuals and references, such as, for example, Sambrook et al., Molecular Cloning: A Laboratory Manual, 2d ed., Cold Spring Harbor Laboratory Press, 1989.

Extension-dependent linker: A linker that cannot substantially bind or hybridize to a primer of interest (such as, a linker-specific primer) because, for example, the linker has no nucleic acid sequence (on either strand) that is complementary to the primer; however, one strand of the linker (for example, the single-stranded portion of the linker) is a template for a binding site for the primer of interest (such as, a linker-specific primer). Thus, a nucleic acid synthesized using at least the linker's template strand (such as, by primer extension) will have a binding site for the primer of interest. Representative examples of extension-dependent linkers are found in U.S. Pat. No. 5,759,822, Lukianov, et al., Bioorganic Chemistry (Russia), 20(6):701-704, 1994; Genome Walker™ Kits User Manual, Protocol #PT 1116-1, Version #PR9Y596, Clontech, Laboratories, Inc. published 10 Nov. 1999; Riley et al., Nuc. Acids Res., 18(10):2887, 1990); Mueller and Wold, Science, 246:246:780-786, 1989; and Arnold and Hodgson, PCR Meth. Appl., 1(1):39-42, 1991).

Nucleic acid molecule: A single- or double-stranded polymeric form of nucleotides, including both sense and anti-sense strands of RNA, cDNA, genomic DNA, and synthetic forms and mixed polymers of the above. A nucleotide refers to a ribonucleotide, deoxynucleotide or a modified form of either type of nucleotide. A “nucleic acid molecule” as used herein is synonymous with “nucleic acid” and “polynucleotide.” The term includes single- and double-stranded forms of DNA or RNA. A polynucleotide may include either or both naturally occurring and modified nucleotides linked together by naturally occurring and/or non-naturally occurring nucleotide linkages.

Nucleic acid molecules may be modified chemically or biochemically or may contain non-natural or derivatized nucleotide bases, as will be readily appreciated by those of ordinary skill in the art. Such modifications include, for example, labels, methylation, substitution of one or more of the naturally occurring nucleotides with an analog, internucleotide modifications, such as uncharged linkages (for example, methyl phosphonates, phosphotriesters, phosphoramidates, carbamates, etc.), charged linkages (for example, phosphorothioates, phosphorodithioates, etc.), pendent moieties (for example, polypeptides), intercalators (for example, acridine, psoralen, etc.), chelators, alkylators, and modified linkages (for example, alpha anomeric nucleic acids, etc.).

The term “nucleic acid molecule” also includes any topological conformation of such molecules, including single-stranded, double-stranded, partially duplexed, triplexed, hairpinned, circular and padlocked conformations. Also included are synthetic molecules that mimic polynucleotides, for instance, in their ability to bind to a designated sequence via hydrogen bonding and other chemical interactions. Such molecules are known in the art and include, for example, those in which peptide linkages substitute for phosphate linkages in the backbone of the molecule.

Unless specified otherwise, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

A “target nucleic acid molecule” (or “target molecule”) is a nucleic acid molecule or population of nucleic acid molecules (such as, genomic DNA) into which at least one integrant has integrated. Thus, a target nucleic acid molecule contains both integrant sequences and non-integrant sequences. Integration of an integrant often will occur when a target nucleic acid molecule is in a native state; for example, contained within the nucleus of a cell. Under native circumstances, various other nucleic acids can also be present with a target nucleic acid molecule. For example, a target nucleic acid molecule can be a specific nucleic acid in a cell (which can include host RNAs and DNAs, as well as other nucleic acid such as viral, bacterial or fungal nucleic acids). In specific examples, a target nucleic acid molecule can be chromosomal DNA or genomic DNA. Purification or isolation of a target nucleic acid molecule, if needed, can be conducted by methods known to those of ordinary skill in the art. For example, purification of genomic DNA can be achieved by using a commercially available purification kit or the like.

Oligonucleotide: A nucleic acid molecule generally comprising a length of 200 or fewer bases. The term often refers to single-stranded deoxyribonucleotides, but it can refer as well to single- or double-stranded ribonucleotides, RNA:DNA hybrids and double-stranded DNAs, among others. In some examples, oligonucleotides are about 10 to about 90 bases in length, for example, 12, 13, 14, 15, 16, 17, 18, 19 or 20 bases in length. Other oligonucleotides are about 25, about 30, about 35, about 40, about 45, about 50, about 55, about 60 bases, about 65 bases, about 70 bases, about 75 bases or about 80 bases in length. Oligonucleotides may be single-stranded, for example, for use as probes or primers, or may be double-stranded, for example, for use in the construction of linkers. An oligonucleotide can be derivatized or modified as discussed in reference to nucleic acid molecules.

Restriction enzyme: A protein (usually derived from bacteria) that cleaves a double-stranded nucleic acid, such as DNA, at or near a specific sequence of nucleotide bases, which is called a recognition site. A recognition site is typically four to eight base pairs in length and is often a palindrome. In a nucleic acid sequence, a shorter recognition site is statistically more likely to occur than a longer recognition site. Thus, restriction enzymes that recognize specific four- or five-base pair sequences will cleave a nucleic acid substrate relatively frequently and may be referred to as “frequent cutters.” Examples of frequent cutting enzymes are shown in Table 1.

Some restriction enzymes cut straight across both strands of a DNA molecule to produce “blunt” ends. Other restriction enzymes cut in an offset fashion, which leaves an overhanging piece of single-stranded DNA on each side of the cleavage point. These overhanging single strands are called “sticky ends” because they are able to form base pairs with a complementary sticky end on the same or a different nucleic acid molecule. Overhangs can be on the 3′ or 5′ end of the restriction site, depending on the enzyme.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of a target protein, and the corresponding cDNA or gene sequence(s), will possess a relatively high degree of sequence identity when aligned using standard methods. This homology will be more significant when the orthologous proteins or genes or cDNAs are derived from species that are more closely related (e.g., human and chimpanzee sequences), compared to species more distantly related (e.g., human and C. elegans sequences).

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene, 73: 237-244, 1988; Higgins & Sharp CABIOS 5: 151-153, 1989; Corpet et al. Nuc. Acids Res. 16, 10881-90, 1988; Huang et al. Computer Appls. in the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. When aligning short sequences (fewer than around 30 nucleic acids), the alignment can be performed using the BLAST short sequences function, set to default parameters (expect 1000, word size 7).

Since MegaBLAST requires a minimum of 28 bp of sequence for alignment to the genome, Pattern Match (available from the Protein Information Resource (PIR) at Georgetown, and at their on-line website) can be optimally used to align short sequences, such as the 15-30 bp, or more preferably about 20 to 22 bp, tags generated in concatamerized embodiments. This program can be used to identify the location of genomic tags within the genome. Another program that can be used to look for perfect matches between the 20 bp tags is ‘exact match,’ which is a PERL computer function that looks for identical matches between two sequences (one being the genome, the other being the 20 bp tag). Since it is expected that there will be single nucleotide polymorphisms within a subset of the identified tags, the exact match program cannot be used to align these tags. Instead, GRASTA (available from The Institute for Genomic Research) will be used, which is a modified FastA code that searches both nucleic acid strands in a database for similar sequences. This program is able to align fragments that contain a one (or more) base pair mismatch(es).

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence remains hybridized to a perfectly matched probe or complementary strand. Conditions for nucleic acid hybridization and calculation of stringencies can be found in Sambrook et al. (In Molecular Cloning: A Laboratory Manual, CSHL, New York, 1989) and Tijssen (Laboratory Techniques in Biochemistry and Molecular Biology—Hybridization with Nucleic Acid Probes Part I, Chapter 2, Elsevier, New York, 1993). Nucleic acid molecules that hybridize under stringent conditions to a protein-encoding sequence will typically hybridize to a probe based on either an entire protein-encoding or a non-protein-encoding sequence or selected portions of the encoding sequence under wash conditions of 2×SSC at 50° C.

Nucleic acid sequences that do not show a high degree of sequence identity may nevertheless encode similar amino acid sequences, due to the degeneracy of the genetic code. It is understood that changes in nucleic acid sequence can be made using this degeneracy to produce multiple nucleic acid molecules that all encode substantially the same protein.

Subject: Living multi-cellular vertebrate organisms, including human and veterinary subjects, such as cows, pigs, horses, dogs, cats, birds, reptiles, mice, rats, and fish.

Vector: A nucleic acid molecule capable of transporting another nucleic acid to which it has been linked. One type of vector is a “plasmid”, which refers to a circular double-stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral (or virally derived) genome. Another category of vectors is integrating gene therapy vectors. Certain vectors are capable of autonomous replication in a host cell into which they are introduced. Some vectors can be integrated into the genome of a host cell upon introduction into the host cell, and thereby are replicated along with the host genome. Some vectors, such as integrating gene therapy vectors or certain plasmid vectors, are capable of directing the expression of heterologous genes which are operatively linked to regulatory sequences (such as, promoters and/or enhancers) present in the vector. Such vectors may be referred to generally as “expression vectors.”

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. The term “comprising” means “including”; hence, “comprising A or B” means including A or B, or including A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Except as otherwise noted, the methods and techniques of the present invention are generally performed according to conventional methods well known in the art and as described in various general and more specific references that are cited and discussed throughout the present specification. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999; each of which is specifically incorporated herein by reference in its entirety.

IV. Methods of Mapping Integration Sites

Methods are disclosed that permit the identification of integrant integration sites. Briefly, a nucleic acid molecule containing at least one integrant (the “target molecule”) is digested with two different restriction enzymes. The first restriction enzyme (N 1) cuts the nucleic acid molecule into numerous fragments. The second restriction enzyme (N2) is selected as described herein to prohibit amplification of an internal fragment of the integrant. Fragments of the target molecule, some of which contain all or part of an integrant, are ligated to an extension-dependent linker (also referred to as an adaptor), which is designed as described herein to substantially inhibit linker-to-linker amplification. Linkered fragments (fragments that contain at least one linker) are then amplified to produce amplification products, which can be cloned without requiring any purification. In particular examples, amplification products containing an integration site junction are sequenced and mapped against known nucleic acid sequences, such as the human genome sequence.

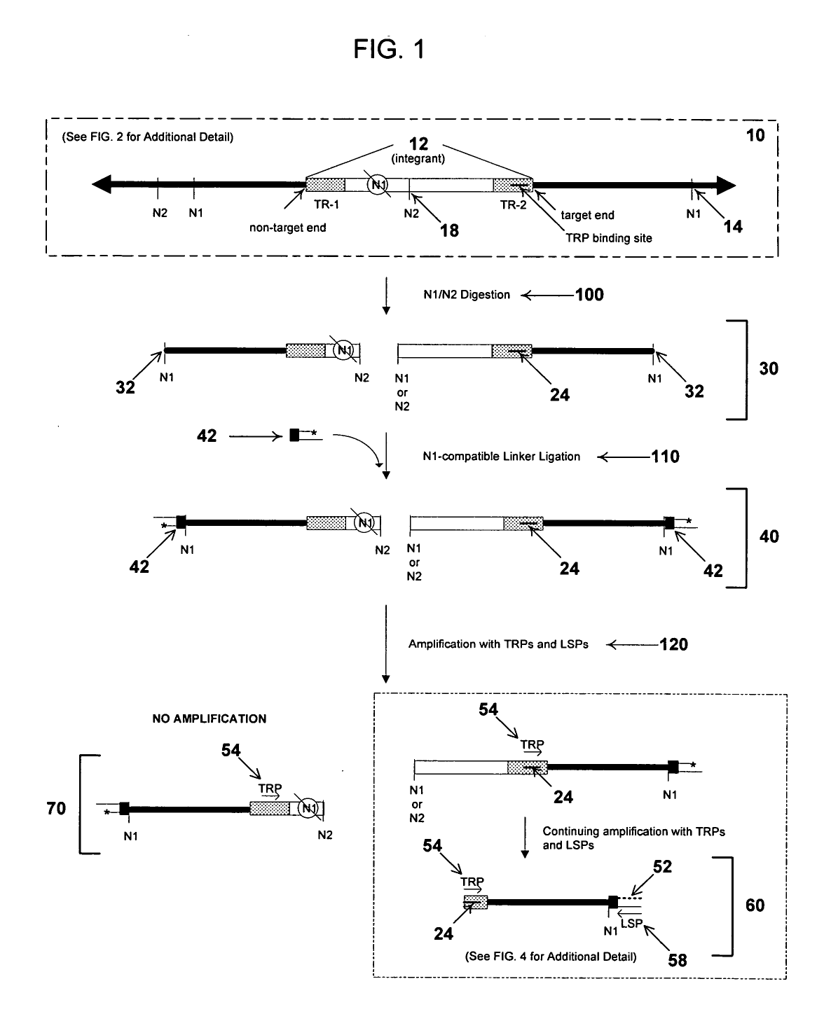

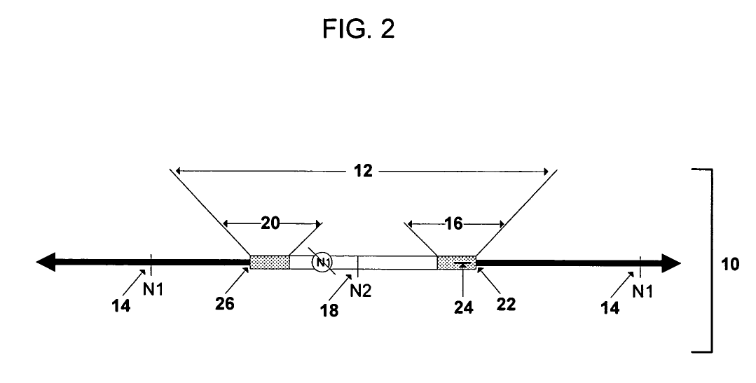

FIG. 1 illustrates one particular method embodiment involving a nucleic acid molecule 10 containing at least one integrant 12 and at least one first restriction site (N1 site) 14, which is cleavable by a first restriction enzyme (N1). As shown in more detail in FIG. 2, the integrant 12 of this representative method includes a first terminal repeat 16, at least one second restriction site (N2 site) 18, which is cleavable by a second restriction enzyme (N2), and a second terminal repeat 20. The first terminal repeat 16 includes a target end 22 and a terminal-repeat-specific primer (TRP) binding site 24, which is complementary to a TRP. The second terminal repeat 20 includes a non-target end 26 and a sequence complementary to the TRP, which is in the same orientation as the TRP binding site 24 in the first terminal repeat 16.

FIG. 1 and FIG. 2 purposefully do not indicate a 5′ or 3′ orientation of any nucleic acid molecule because the described methods work equally to analyze the 3′ or 5′ integration junctions. Each “end” of an integrant 12 is substantially the same as the other end to the extent that each end includes a same-orientation sequence (located in the terminal repeat) that can stably bind a TRP; that is, the first terminal repeat 16 includes a TRP binding site 24, and the second terminal repeat 20 includes a sequence complementary to the TRP. Thus, the non-target end of an integrant can become the target end (and visa versa) by re-designing the TRP so that its extension (for example, by DNA polymerase) is toward (rather than away from) the end of the integrant desired to be amplified (that is, the target end). In this manner, the extension product of the TRP will predominantly include non-integrant, flanking sequence (rather than predominantly internal integrant sequences).

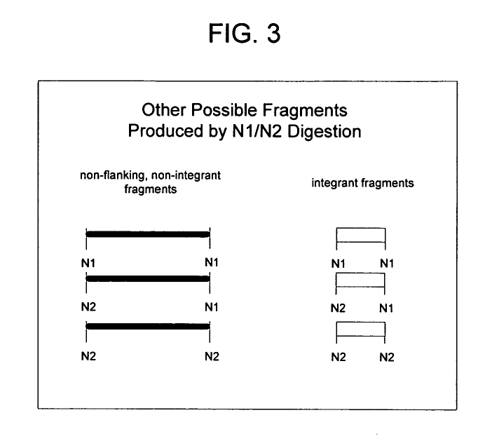

As further illustrated in FIG. 1, the nucleic acid molecule 10 is digested 100 with N1 and N2 (concurrently or in sequence, without preference to the order of digestion) to produce a population of nucleic acid fragments 30 (though it is noted that not all possible fragments are shown in FIG. 1). Fragments containing integrant nucleic acid sequences together with non-integrant flanking nucleic acid sequences (referred to as “integration junction fragments”) are of particular use in the disclosed methods. Other possible nucleic acid fragments that may result from digestion with N1 and N2, but which are not integration junction fragments, are shown in FIG. 3. Fragments such as those shown in FIG. 3 are not substantially amplified in the disclosed methods, as discussed in more detail below.

N2 is selected to cleave the integrant 12 so there are no N1 sites between the non-target end 26 and the N2 site 18 closest to the non-target end 26. Methods of selecting a restriction enzyme for such a purpose are well known in the art. For example, an ordinarily skilled artisan may generate (or obtain) a restriction map of an integrant, which shows the relative positions of any known restriction enzyme sites in an integrant sequence. With such a map, one can determine which enzymes are suitable for use as N1 or N2 as described herein.

With continued reference to FIG. 1, at least some fragments 30 produced by digestion with N1 and N2 contain “N1 ends” 32, such as overhanging ends or blunt ends, which are produced by cleavage of the nucleic acid molecule 10 with N1. An extension-dependent linker 42 is ligated 110 to at least some of the N1 ends 32 to produce a population of linkered fragments 40. Extension-dependent linker 42 is partially double stranded and partially single stranded to form an overhang. In some embodiments, such as the illustrated embodiment, the overhang is a 5′ overhang.

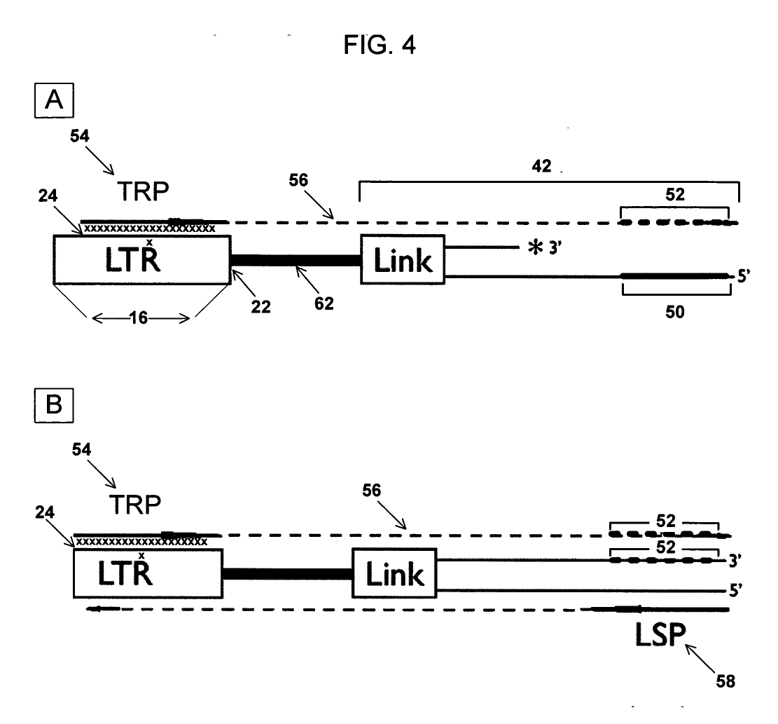

As shown in more detail in FIG. 4A, extension-dependent linker 42 provides a template 50 for a linker-specific primer (LSP) binding site 52. Thus, when a TRP 54 is extended (illustrated with a dashed line in FIG. 4A) to produce an extension product 56 during the first (and subsequent) rounds of amplification 120, a LSP binding site 52 is produced in the extension product 56. In subsequent rounds of amplification 120 (as detailed in FIG. 4B), an extension product 56 may serve as a template and bind a LSP 58. In accordance with in vitro amplification principles, which are well known in the art, the nucleic acid sequence between the TRP binding site 24 (in the integrant) and the LSP binding site 52 (in the linker portion of an extension product 56) can be amplified. A product of the foregoing amplification will be an integration junction fragment (fragment 60 as shown in FIG. 1) and contains a copy of the target end 22 and nucleic acid sequences flanking the target end.

As one of skill in the art will recognize, fragments such as those shown in FIG. 3 and an integration junction fragment containing a non-target end 70 will not be substantially amplified in the disclosed methods because such fragments either cannot (or are unlikely to) bind any pair of primers (for example, two TRPs, two LSP, or a TRP and an LSP) in the proper orientation for amplification.

An integration site may be identified from an amplified integration junction fragments containing either the 3′ or the 5′ end of an integrant. A target end is the particular end of an integrant from which non-integrant, flanking nucleic acid sequence is (or is to be) obtained in particular embodiments. A target end may be located at the 3′ or the 5′ end of an integrant. In particular embodiments, a target end is located at the 3′ end of an integrant, in which case 3′ flanking nucleic acid sequences are amplified and sequenced. In other embodiments, a target end is the 5′ end of an integrant, in which case 5′ flanking nucleic acid sequences are amplified and sequenced.

The disclosed methods may, but need not, be performed in one or a few days. Particular method embodiments can identify substantial numbers of integration sites in as few as about 14 days, such as no more than about 10 days, no more than about 7 days, no more than about 5 days, or no more than about 4 days (as opposed to the weeks or months necessary to identify comparable numbers of integration sites by other technologies, such as that described in Schroder et al., Cell, 110:521-529, 2002). Other disclosed methods avoid selection bias, and minimize amplification and cloning biases. In still other of the disclosed methods, greater than about 70%, about 80%, about 85%, about 90%, about 95%, or about 98% of amplification products represent integration junction site fragments.

Particular elements of embodiments of the disclosed methods are discussed in more detail in the subsections that follow.

1. Nucleic Acid Molecules

Nucleic acid molecules useful in the disclosed methods include any nucleic acid molecule capable of containing at least one integrant. Such nucleic acid molecules include, without limitation, genomic DNA (including chromosomal DNA), plasmid DNA, yeast artificial chromosomes (YACs), bacterial artificial chromosomes (BACs), P1-derived artificial chromosomes (PACs), cosmids or fosmids. In some examples, a nucleic acid molecule is genomic DNA. Genomic DNA may be obtained, for example, from one or more cells by methods known in the art (for example, kits for this purpose are commercially available from Promega, Roche Biochemical, Bio-Nobile, Brinkmann Instruments, BIOLINE, MD Biosciences, and numerous other commercial suppliers; see, also, Sambrook et al., Molecular Cloning: A Laboratory Manual, New York: Cold Spring Harbor Laboratory Press, 1989; Ausubel et al., Current Protocols in Molecular Biology, New York: John Wiley & Sons, 1998). Genomic DNA can also be obtained from any biological sample that may be obtained directly or indirectly from a subject, including whole blood, plasma, serum, tears, bone marrow, lung lavage, mucus, saliva, urine, pleural fluid, spinal fluid, gastric fluid, sweat, semen, vaginal secretion, sputum, fluid from ulcers and/or other surface eruptions, blisters, abscesses, and/or extracts of tissues, cells or organs. The biological sample may also be a laboratory research sample such as a cell culture supernatant. The sample is collected or obtained using methods well known to those ordinarily skilled in the art.

In specific examples, genomic DNA is eukaryotic genomic DNA. Genomic DNA can be obtained from an organism (or cells thereof) for which the sequence of genomic DNA is substantially known, including for instance, human (Homo sapiens), mouse (Mus musculus), rat (Rattus norvegicus), or zebrafish (Danio rerio), Caenorhabditis elegans, Drosophila melanogaster, or Anopheles gambiae genomic DNA.

A target nucleic acid molecule useful in the disclosed methods includes one or more integrants. The integrants contained in a nucleic acid molecule may be the same or different. The actual number of integrants contained in a nucleic acid will depend on various factors; for instance, the nature of the integrant, the nature of the nucleic acid molecule, the capacity of the nucleic acid molecule to assimilate integrants, the presence or absence of facilitators or inhibitors of integration, or the total number of integrants exposed to the nucleic acid. In some instances, a nucleic acid molecule, such as, a single chromosome, all or some of the genomic DNA from a single cell, a BAC, a YAC, or cosmid, may contain one, two, five, ten, fifteen or more integrants. In other instances, a nucleic acid molecule, includes a collection of nucleic acid molecules (typically, same-type nucleic acid molecules) isolated from a population of cells; for example, total genomic DNA isolated from at least about 10³, 10⁴, 10⁵, 10⁶ or even more cells. In the situation where the nucleic acid molecule is isolated from a cell population, the total number of integrants available for identification using the disclosed methods can be at least 100, at least 200, at least 500, at least 750, at least 1000, at least 1500, at least 2000 or even more integrants.

Different types of integrants in the same target molecule (for example, HIV-1 and MLV in human genomic DNA) may be simultaneously identified using the disclosed methods by including appropriate TRPs specific for each type of integrant.

2. Integrants

An integrant is a nucleic acid molecule that integrates (or inserts) itself into another nucleic acid molecule (which may be referred to as a target nucleic acid molecule). The mechanism by which such insertion occurs is not of particular importance to the disclosed methods, for example, integration of an integrant may occur naturally (such as, as a result of infection of an individual or a cell by an integrant) or may be engineered (for example, using molecular techniques known in the art to insert an integrant into a target nucleic acid molecule). For the purposes of this disclosure, it is the fact that the integrant is integrated into a nucleic acid molecule that is of consequence.

Integrants may include, for example, viruses, transposons, transgenes, integrating gene therapy vectors, and fragments of any of these. In particular embodiments, an integrant is a virus (such as a DNA virus, a retrovirus, or other RNA virus). Representative integrating viruses are well known in the art (see, for example, the viral genome database available on the National Center for Biotechnology Information (NCBI) website, which includes more than 1500 viral genomic sequences and characteristics of such viruses). Specific examples of integrating DNA viruses include, without limitation, adeno-associated viruses. Specific examples of retroviruses include, without limitation, murine leukemia virus, human immunodeficiency virus 1 (HIV-1), human spumavirus, lentiviruses, Rous sarcoma virus, avian sarcoma virus, mouse mammary tumor virus (MMTV), gross mouse leukemia virus, avian leukosis virus, bovine leukemia virus, Walley dermal sarcoma virus, human foamy virus (HFV), simian immunodeficiency virus (SIV), and murine sarcoma virus (MSV).

Other integrants are integrating gene therapy vectors. Such vectors may be derived, for example, from integrating viruses (discussed above) or transposable elements, such as the Sleeping Beauty transposon. For example, virally derived integrating gene therapy vectors may be engineered from a particular viral strain to affect a particular characteristic of the virus; for instance, to cause increased expression of a gene transferred by the vector, to develop improved packaging and more effective and/or controlled gene delivery, to target appropriate cell populations for gene transfer, and/or to selectively minimize or repress immune response of the host organism (see, for instance, reviews by Lipps et al., Gene, 304:23-33, 2003; Lundstrom, Trends Biotechnol., 21(3):117-122, 2003; Oupicky and Diwadkar, Curr. Opin. Mol. Ther., 5(4):345-350, 2003; Owens, Curr. Gene Ther., 2(2):145-159, 2002; Pandya et al., Expert Opin. Biol. Ther., 1(1):17-40, 2001; Carter and Samulski, Int. J. Mol. Med., 6(1):17-27, 2000; Strayer, J. Cell. Physiol., 181(3):375-384, 1999). Such engineering may involve, among other things, deletion, or other mutation, of viral genes, and/or addition of heterologous genes to the viral genome.

An integrant useful in the disclosed methods includes (among other things) a first and a second terminal repeat. Terminal repeats are substantially similar nucleic acid sequences that are present at both ends of an integrant. Terminal repeats include, for example, long terminal repeats (LTRs) and short terminal repeats, of a sort typically found in retroviruses and other retroelements (such as, retrotransposons), and in many integrating gene therapy vectors. The nucleic acid sequences of terminal repeats that flank the same integrant can be at least 80%, at least 90%, at least 95%, at least 99% or even 100% identical. In particular, a second terminal repeat, as disclosed herein, includes a sequence capable of stably binding a TRP, which sequence is in the same orientation as the TRP binding site in the first terminal repeat. The lengths of terminal repeats may vary considerably among different integrants; for example, terminal repeats (such as, LTRS) may range from several hundred nucleotides to more than a thousand nucleotides. The nucleic acid sequences of the first and second terminal repeats of the disclosed methods will have the same orientations. For example, if a portion of one strand of a terminal repeat reads 5′-GTCAT-3′, then the same strand of the paired terminal repeat in the same orientation would also read 5′-GTCAT-3′.

A first terminal repeat of an integrant further includes, without limitation, a TRP binding site, which is complementary to a TRP (for example, a representative TRP binding site 24 and TRP 54 are shown in FIGS. 4A and 4B). A TRP binding site can be any number of nucleotides, typically contiguous nucleotides, to which a TRP stably binds. For example, a TRP binding site may be 10, 15, 20, 25, 30 or 50 nucleotides or more in length. A TRP binding site typically will have a nucleic acid sequence complementary to a TRP. A TRP binding site may be located on either strand of an integrant. In specific examples, a TRP binding site is located no more than about 500 base pairs, no more than about 300 base pairs, no more than about 200 base pairs, or no more than about 100 base pairs from the target end of an integrant.

A TRP stably binds a TRP binding site. A TRP has the general characteristics of a “primer,” which have been previously described.

3. Digestion of a Nucleic Acid Molecule(s)

In the disclosed methods, nucleic acid molecules comprising at least one integrant are digested (or cut) into fragments using two different restriction enzymes, referred to herein as a first restriction enzyme (or N1) and a second restriction enzyme (or N2), respectively. The foregoing terminology does not imply any order in which the particular enzymes may be used in the disclosed methods, and in some embodiments the enzymes are used concomitantly. The contemplated restriction enzymes may cleave the nucleic acid molecule to leave blunt ends or overhanging (also called, sticky) ends. In some embodiments, N1 and N2 leave overhanging ends. Restriction enzyme digests may be performed concomitantly (at the same time; also called, a co-digestion) or successively (such as, a sequential digestion).

In some method embodiments that include concomitant digestions, N1 and N2 ends are incompatible with each other; for example, an N1 end may not be directly ligated to an N2 end to form a single nucleic acid molecule. In method embodiments including successive digestions, N1 and N2 ends may be either compatible (for example, both leaving blunt ends, or both leaving mutually compatible sticky ends) or incompatible. In particular methods including successive restriction enzyme digestion wherein N1 and N2 have compatible ends, N1 digestion is first performed, followed by linker ligation (described below), followed by removal of unbound linkers, followed by N2 digestion.

The N1 restriction enzyme used in methods disclosed herein recognizes a first restriction site (N1 site) that is typically no more than five contiguous base pairs in length; for example, N1 recognizes four contiguous base pairs or five contiguous base pairs. As such, N1 may be referred to as a “frequent cutter.” In some examples, N1 recognizes a non-degenerate restriction site having a sequence of only T and A nucleic acids. Such restriction enzymes are known in the art (see, for example, Life Science Catalog 2002, Promega Corporation, Madison, Wis., pages 88-122; 2002-03 Catalog & Technical Reference, New England Biolabs, Inc., Beverly, Mass., pages 13-65). Examples of restriction enzymes useful as N1 include those shown in Table 1. In particular examples, N1 is MseI, RsaI, TaqI, Tri1I or RsaI.

A target nucleic acid molecule will contain at least one N1 site that is not located within an integrant. One or more N1 site(s) may, but need not, be located within an integrant sequence. If an N1 site is located within an integrant, N1 should not cut between the TRP binding site 24 (see, for example, FIG. 2) and the target end 22 (see, for example, FIG. 2).

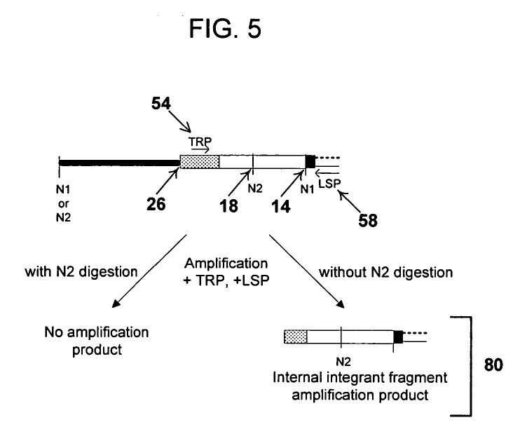

The second restriction enzyme (N2) used in the methods disclosed herein is useful to inhibit amplification of an internal fragment of the integrant (see, for example, internal integrant fragment 80 in FIG. 5). An internal integrant fragment contains no non-integrant flanking nucleic acid sequence and, therefore, is not useful to identify integration sites. Moreover, because an internal fragment is likely to be amplified for substantially all integrants in a nucleic acid molecule, internal integrant fragments may make up a substantial percentage of the amplification products. This is disadvantageous because it obscures the desired integration junction fragments in subsequent analysis.

N2 is selected based on the integrant's nucleic acid sequence. If the integrant contains no N1 sites, N2 is selected to cut the integrant at a specific restriction site between the non-target end 26 and the TRP binding site 24 (with reference to FIG. 2). If the integrant contains one or more N1 sites, N2 is selected to cut the integrant between the non-target end 26 and the integrant N1 site 14 that is closest to the non-target end (for instance, with reference to FIG. 5). In summary, there should not be an intervening N1 site between the non-target end and the N2 site in the integrant that is closest to the non-target end. N2 also should not cut between the TRP binding site 24 (see, e.g., FIG. 2) and the target end 22 (see, e.g., FIG. 2). N2 may recognize any restriction site (or sites) as long as such site is located as described herein. As a result of selection of N2 as described herein, the integrant portion of an integration junction fragment containing a non-target end (fragment 70 as shown in FIG. 1) will have a N2 end. In some method embodiments, an N1-compatible, extension-dependent linker will not substantially ligate to an N2 end if N1 ends and N2 ends are incompatible.

In specific embodiments, N2 cuts a target nucleic acid molecule comprising at least one integrant no more frequently than does N1. In specific embodiments, N2 cuts a nucleic acid molecule less frequently than does N1. For example, in some embodiments, N2 has a recognition site of six or more consecutive nucleotides. Representative restriction enzymes useful as N2 are known in the art (see, for example, Life Science Catalog 2002, Promega Corporation, Madison, Wis., pages 88-122; 2002-03 Catalog & Technical Reference, New England Biolabs, Inc., Beverly, Mass., pages 13-65). In particular examples, N2 is PstI, Bgl II, or EcoRI.

Because non-integrant flanking sequences of the target molecule are not known, it is possible that an N2 site will be closer to a target end than an N1 site. In this event, that particular target end will not be represented in the resultant integration junction fragment library. To minimize this possibility, it is advantageous for N2 to cut the target nucleic acid molecule less frequently than N1 (as described previously). In addition (or alternatively), the user may elect to perform the disclosed methods using a different N2 enzyme, or using a different combination of N1 and N2.

Restriction enzyme digestions are performed under conditions commonly known in the art. Typically, each restriction enzyme has preferred reaction conditions, which are provided to the user by the manufacturer. Factors that may be considered for any particular enzyme include reaction temperature, buffer pH, enzyme cofactors, salt composition, ionic strength and/or stabilizers. A representative restriction enzyme reaction is performed in a volume of approximately 20 μl on 0.2-1.5 μg of substrate DNA using a 2- to 10-fold excess of enzyme over DNA, based on unit definition. Such conditions can be scaled up for larger amounts of substrate DNA. In particular examples, about 1 μg of genomic DNA is incubated with at least about 10 units of at least one restriction enzyme at 37° C. for about 2 hours in a buffer(s) supplied by the manufacturer. A restriction enzyme digestion, optionally, may be terminated by heating the reaction mixture to a temperature that will inactivate the restriction enzyme(s), such as heating to at least about 65° C.

An ordinarily skilled artisan will appreciate that some digests using multiple restriction enzymes that have different optimal reaction conditions may be satisfactorily performed, for example, using a buffer that is compatible with each of the multiple enzymes, and/or by making adjustments in the number of units of enzyme used. Such buffers may be different from the buffers useful for reactions using any one of the restriction enzymes alone. Buffers useful for multiple restriction enzymes digestions are known in the art (see, for example, the Restriction Enzyme Resource available on the Promega Internet site under the “Technical Resources” link and “Guides” sublink; and the Double Digest technical information available on the New England Biolabs Internet site under the “Tech Resource,” “Technical Literature,” “Restriction Enzymes,” “NEBuffer System” thread). Rather than identifying a compatible buffer, it is also acceptable to perform sequential reactions in which, for example, additional buffer or salt is added to a reaction before the second enzyme, or each digest is performed sequentially using the optimal buffers with a DNA precipitation or purification step after the first digest.

Following restriction enzyme digestion, a target nucleic acid molecule will have been cleaved into at least two nucleic acid fragments, at least 100, at least 1000, at least 5000, at least 10,000 or even more nucleic acid fragments. Certain fragments will have only N1 ends, other fragments will have one N1 end and one N2 end (such as, a fragment with a 5′ N1 end and a 3′ N2 end, or a fragment with a 5′ N2 end and a 3′ N1 end), and still other fragments will have only N2 ends (for exemplar fragments, see FIGS. 1 and 3). Nucleic acid fragments will be various sizes depending, in part, upon how often N1 and N2 restriction sites occur in the nucleic acid molecule. For example, nucleic acid fragments up to about 3000 base pairs, up to about 2000 base pairs, up to about 1000 base pairs, up to about 500 base pairs, up to about 250 base pairs, up to about 100 base pairs, up to about 30 base pairs can be expected under restriction enzyme digestion conditions disclosed herein. In other examples, 80%, 90%, 95%, or 98% of the nucleic acid fragments in a population are of the lengths just described. In yet other examples, a population of nucleic acid fragments has an average length of about 500 bases pairs, about 250 base pairs, about 100 base pairs, or about 70 base pairs, following restriction digestion step(s) of the disclosed methods.

Because a target nucleic acid molecule contains at least one non-integrant N1 site and an integrant contains at least one N2 restriction site, the target end and the non-target end of an integrant will generally be located on separate integration junction fragments. Each such integration junction fragment, thus, contains an integrant portion and a portion of non-integrant flanking sequence.

In embodiments where the target end is the 5′ end of the integrant, N2 will be selected so that after N2 cleavage the integrant portion of the 3′ integration junction fragment either (i) cannot substantially bind an N1-compatible extension-dependent linker, or (ii) has been cleaved from an N1-compatible extension-dependent linker that may have been ligated to the integrant portion. In embodiments where the target end is the 3′ end of the integrant, then N2 will be selected so that after N2 cleavage the integrant portion of the 5′ integration junction fragment either (i) cannot substantially bind an N1-compatible extension-dependent linker, or (ii) has been cleaved from an N1-compatible extension-dependent linker that may have been ligated to the integrant portion.

4. Amplification Primers

The disclosed methods involve in vitro amplification of at least a portion of integration junction fragments. In vitro amplification (such as, PCR) involves a pair of primers that are annealed to sites at or near each end (and on opposite strands) of the sequence to be amplified. In the disclosed methods, the sequence to be amplified is at least a part of an integration junction fragment, which includes the junction between the integrant and the non-integrant flanking nucleic acid sequence. At least some of the sequence of the integrant portion of an integration junction fragment (such as, a terminal repeat) is known with sufficient detail to design primers that can stably bind such sequence (such as, a TRP). An integrant-binding primer can be extended across a target end and into the non-integrant nucleic acid sequence flanking the target end.

Flanking, non-integrant sequence of an integration junction fragment is presumed to be unknown; therefore, it is not feasible to design a primer that can bind the non-integrant, flanking sequence for purposes of amplification of all or part of an integration junction fragment. To overcome this limitation, a linker of known (or partially known) sequence is ligated to the unknown end of an integration junction fragment to be amplified. One or more linker-specific primers (LSP) then may be designed to stably bind to the linker. Together, an LSP (binding to one strand of the linker) and an integrant-binding primer (such as, a TRP) (binding to the opposite strand in the integrant) are used to amplify the nucleic acid sequence between the two primer binding sites, which includes the target end of the integrant integration site.

A primer useful in the disclosed methods (for example, an LSP or an integrant-binding primer) is an oligonucleotide, whether occurring naturally as in a fragment obtained from purified restriction digest, or produced synthetically, which is capable of acting as a point of initiation of extension product synthesis when placed under conditions in which synthesis of a primer extension product which is complementary to a nucleic acid strand is induced (for example, in the presence of nucleotides and of an inducing agent such as DNA polymerase and at a suitable temperature and pH). The primer is preferably single stranded for maximum efficiency in amplification, but may alternatively be double stranded. If double stranded, the primer is often first treated (denatured) to separate its strands before being used to prepare extension products.

Primers are typically short nucleic acid molecules, for instance DNA oligonucleotides 10 nucleotides or more in length. The exact lengths of the primers will depend on many factors, including temperature of the annealing reaction, source of primer and the use of the method. Representative primers may be about 15, 20, 25, 30 or 50 nucleotides or more in length. Primers can be annealed to a complementary target DNA strand by nucleic acid hybridization to form a hybrid between the primer and the target DNA strand. Optionally, the primer then can be extended along the target DNA strand by a DNA polymerase enzyme. Primer pairs can be used for amplification of a nucleic acid sequence, for example, by the polymerase chain reaction (PCR) or other in vitro nucleic acid amplification methods known in the art. For use in in vitro amplification methods, the primer must, at least, be sufficiently long to prime the synthesis of extension products in the presence of the inducing agent.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). Amplification primer pairs (for instance, for use with in vitro amplification) can be derived from a known sequence, for example, by using computer programs intended for that purpose such as Primer (Version 0.5, © 1991, Whitehead Institute for Biomedical Research, Cambridge, Mass.).

One of ordinary skill in the art will appreciate that the specificity of a particular primer increases with its length. Thus, for example, a primer comprising 30 consecutive nucleotides complementary to a nucleic acid will anneal to the target sequence with a higher specificity than a corresponding primer of only 15 nucleotides. Thus, in methods where specificity is a consideration, primers can be selected that comprise at least 20, 23, 25, 30, 35, 40, 45, 50 or more consecutive nucleotides complementary to the target sequence.

5. Linkers, Linker Ligation and Linkered Integration Junction Fragments

In the disclosed methods, the non-integrant portion of an integration junction fragment is typically unknown. As discussed above, a linker of known (or partially known) sequence may be ligated to the unknown end of an integration junction fragment to overcome this limitation and enable amplification of the integration junction fragment.

A linker is an at least partially double-stranded nucleic acid molecule, for example a DNA sequence, which is capable of being ligated to another double-stranded nucleic acid molecule, such as nucleic acid fragment produced by restriction enzyme digestion of a target nucleic acid sequence, including for example genomic DNA or plasmid DNA. Linkers may be produced, for example, by annealing two synthetic oligonucleotides that have, at least in part, complementary sequences. Representative oligonucleotides, which may be annealed to form one exemplar linker useful in the disclosed methods, are provided in SEQ ID NOs: 1 and 2. The individual nucleic acid strands of a linker need not be the same length, and may range independently in length as described previously for oligonucleotides. Where the two strands are not the same length, the resultant linker will be only partially double-stranded, and will have 3′ or 5′ overhang(s) on one end or both.

One or more nucleotides in one or both strands of a linker may be modified as described for nucleic acid molecules. In some examples, the 3′-terminal nucleotide is modified to substitute a chemical group that will serve to block 3′ extension of the strand containing that modified nucleotide, such as substitution of an amine group for the 3′ terminal hydroxyl group (see, for example, linker 42 in FIG. 4).

A linker may have either or both a 5′ and/or 3′ overhang, for example, to form one or more “sticky” ends compatible with one or more restriction enzymes, which is useful for ligating the linker to a second nucleic acid digested with one or more such restriction enzymes. The sequence of one or both strands of a linker may, optionally, include primer binding sites or restriction enzyme recognition sites, for example, to facilitate in vitro amplification and/or cloning. Overhang(s) also provide for the “extension dependence” of representative linkers.

Linker (or ligation)-mediated PCR (LM-PCR) has been previously described and is well known in the art (see, for example, Mueller and Wold, Science, 246:780-786, 1989; Garrity and Wold, Proc. Natl. Acad. Sci. USA, 89:1021-1025, 1992). Some applications of LM-PCR may produce undesirable amplicons (such as, non-flanking genomic fragments having linkers on either end) as a result of linker-to-linker amplification. Thus, a variety of specialized linkers are known in the art and can be designed based on the teachings herein, which suppress linker-to-linker amplification in LM-PCR. Such linkers are referred to herein as “extension-dependent linkers.”

Extension-dependent linkers have one strand that serves as a template for a primer binding site, but, importantly, such linkers do not themselves include a binding site for that primer. Examples of extension-dependent linkers include vectorette units, boomerang units, and linkers useful for the GenomeWalker™ method (see, for example, Hui et al., Cell. Mol., Life Sci., 54:1403-1411, 1998; Riley et al., Nuc. Acids Res., 18:2887-2890, 1990), splinkerette units (see, for example, Hui et al., Cell. Mol., Life Sci., 54:1403-1411, 1998; Devon et al., Nuc. Acids Res., 23:1644-1645, 1995; U.S. Pat. No. 5,759,822, Lukianov, et al., Bioorganic Chemistry (Russia), 20(6):701-704, 1994; GenomeWalker™ Kits User Manual, Protocol #PT1116-1, Version #PR9Y596, Clontech, Laboratories, Inc., published 10 Nov. 1999).

In the disclosed methods, extension-dependent linkers have one end that may be ligated to (is compatible with) nucleic acid fragments having N1 ends. With reference to one embodiment shown in FIG. 4, an extension-dependent linkers 42 may ligate to the non-integrant end of an integration junction fragment and provide a template 50 for a LSP binding site 52. Copying of template 50 by extension of a TRP 54 bound to an integrant portion of a linkered integration junction fragment (such as a TRP binding site 24) produces an extension product 56, which includes a LSP binding site 52. Such extension product 56 may serve an in vitro amplification template in combination with its complementary strand of the integration junction fragment in the presence of TRPs 54 and LSPs 58 to amplify the portion of an integration junction fragment between the TRP and LSP primer binding sites (see, for example, fragment 60 in FIGS. 1 and 4). The amplified portion of an integration junction fragment between the TRP and LSP primer binding sites may be referred to as an integration junction amplicon.

Extension-dependent linkers are ligated to nucleic acid fragments, such as integration junction fragment, using methods known in the art. The ligase used can depend on the target nucleic acid molecule. For example, if the target nucleic acid molecule is DNA, representative ligases include E. coli DNA ligase, T4 DNA ligase, Taq DNA ligase, and AMPLIGASE. DNA ligase catalyzes the formation of a phosphodiester bond at a break in a DNA chain. DNA ligase requires a free 3′ hydroxyl group and a 5′ phosphoryl group. The ligase used can determine the reagents needed to effect the ligation reaction. In particular examples, the ligase reaction includes ATP or NAD as an energy source, Mg⁺⁺, or combinations thereof. Typically, the ligase manufacturer will provide the appropriate buffer(s) and instructions for performing a ligase reaction. In one example, a ligase reaction involves high-concentration T4 DNA ligase (New England Biolabs), between about 100-500 μmole (such as 300 μmole) extension-dependent linker, about 5 ng or less (such as, 2.5 ng or 1 ng) of digested genomic DNA, ligase buffer provided by the ligase manufacturer, in a final volume of between about 15 μl and about 50 μl for 2 hours or more at room temperature.

6. Amplification, Cloning and Sequencing of Integration Junction Amplicons

As appreciated by those of ordinary skill in the art, PCR enables amplification of a nucleic acid sequence which lies between two regions of known nucleotide sequence (see, for example, Mullis et al., U.S. Pat. Nos. 4,683,202 and 4,683,195; Mueller et al., U.S. Pat. No. 5,599,696). Oligonucleotides complementary to known 5′ and 3′ sequences flanking the nucleic acid to be amplified (the target or template) serve as “primers,” for instance TRPs and LSPs. In the PCR, double-stranded target nucleic acid is first melted (dissociated) to separate the two strands. The oligonucleotide primers complementary to the known 5′ and 3′ portions of the segment which is desired to be amplified are then annealed to the target nucleic acid. The portions of the nucleic acid target where the primers anneal serve as starting points for the synthesis of new complementary nucleic acid strands (extension products). This process utilizes an added DNA or RNA polymerase, most often Taq DNA polymerase, although other appropriate DNA polymerases are known. The enzymatic synthesis of the complementary nucleic acid strands is known as “primer extension.” The orientation of the 5′ and 3′ primers with respect to one another is such that the 5′ to 3′ extension product from each primer contains, when extended far enough, the sequence which is complementary to the other primer. Thus, each newly synthesized nucleic acid strand becomes a template for synthesis of yet another nucleic acid strand beginning with the opposite primer. Repeated cycles of melting, annealing of primers, and primer extension lead to a (near) doubling of nucleic acid strands with each cycle. Each new strand contains the sequence of the target nucleic acid beginning with the sequence of the first primer and ending with the sequence of the second primer.

In some embodiments of the disclosed methods, nested PCR may be performed. Nested PCR is a technique known in the art (see, for example, PCR: Essential Data, ed. by C. R. Newton, West Sussex, United Kingdom: John Wiley & Sons, 1995; PCR: Essential Techniques, ed. by C. R. Newton, West Sussex, United Kingdom: John Wiley & Sons, 1996; Cantor and Smith, Genomics, New York: John Wiley & Sons, 1999, page 105). Nested PCR can be useful to increase the specificity and sensitivity of a PCR reaction. Briefly, nested PCR employs two pairs of PCR primers in sequential reactions to amplify a particular nucleic acid sequence, such as an integration junction fragment. The first primer pair produces a first amplification product as described above in the general description of the PCR process. The second pair of primers (also, called “nested primers”) bind within the first amplification product and produce a second amplification product that will be at least somewhat shorter than the first amplification product. This technique is based on the concept that if the wrong sequence is amplified using the first primer set, the probability is very low that it would also bind and be amplified using the nested primers. Exemplar nested primers useful in some embodiments are shown in SEQ ID NOs: 4, 6 and 8.

In some embodiments, it is useful to keep amplicons reasonably short, which allows for shorter polymerase extension times in the PCR cycles (typically, extension time has a linear relationship to time of reaction). Under these circumstances, it is less likely that a polymerase will initiate incorrect or spurious extension reactions, thereby improving specificity of a PCR reaction. Moreover, amplification of shorter fragments is known to reduce PCR bias against large fragments and allow the read-through of most fragments in a single sequence pass (see, for example, Cheung and Nelson, Proc. Natl. Acad. Sci. USA, 93:14676-14679, 1996, which showed a bias against amplification of large genomic DNA fragments using non-specific primers). By reducing such possible PCR bias, the resultant clones are more representative of all integration sites in a given target nucleic acid. In particular examples of the disclosed methods, integration junction fragments (or the portion thereof that is to be amplified) present in an amplification reaction may have an average length of about 500 bases pairs, about 250 base pairs, about 100 base pairs, or about 70 base pairs.

Cloning of integration junction amplicons into any vector can be performed using any method known in the art. As discussed above, extension-dependent linkers may be designed to provide restriction sites useful for cloning. Of particular use in the disclosed methods is “shot-gun cloning.” In shot-gun cloning, a mixture of different nucleic acid fragments (such as, DNA fragments or, more particularly, PCR amplicons) is cloned without purification into a receiving vector. In some examples of the disclosed methods, integration junction amplicons are shot-gun cloned into a vector without prior purification of the amplicons.

Useful cloning vectors and cloning protocols are well known to those of ordinary skill in the art (see, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999).

For example, “TA cloning” takes advantage of the terminal transferase activity of some DNA polymerases, such as Taq polymerase (see, for example, Marchuk et al., Nuc. Acids. Res., 19:1154, 1991). Terminal transferase activity of a polymerase results in a single, 3′-A overhang to each end of a PCR product. These 3′ overhangs make it possible to clone a PCR product directly (that is, without prior restriction digestion) into a linearized cloning vector with single, 3′-T overhangs. The complementary overhangs of the cloning vector and PCR product can be ligated to form a single nucleic acid molecule. Representative TA cloning vectors include, for example, pGEM-T (Promega), pTA Plus, pTA (Genetech), and pCRII T-A (Invitrogen).

To avoid a separate ligation step, TOPO® technology (Invitrogen) may be used. In this cloning method, a commercially available pre-linearized vector is provided. The vector has DNA topoisomerase I covalently bound to each 3′ end. Topoisomerase I, which functions as both a restriction enzyme and a ligase, cleaves itself from the vector leaving an end compatible with the PCR fragment and then joins the compatible PCR fragment. A typical reaction is performed at room temperature and is complete in about 5 minutes.

Optionally, some embodiments involve concatenated tags of integration junction amplicon that contain about 20 bp of sequence adjacent to each extension-dependent linker. Since only a small amount of sequence (10-30 bp, more preferably about 20-22 bp, and most preferably 21 bp) is needed to determine the location of each integrant within the target nucleic acid molecule, concatemers of amplicon tags will permit about 30 putative integration sites to be identified from a single sequencing pass; thus, accelerating the sequencing of putative integration sites. The about 20-bp tag is produced by including a consensus recognition site for a Type IIs restriction endonuclease, such as MmeI, in the sequence of the extension-dependent linker. MmeI is recommended because it cuts the farthest away from its own recognition sequence, compared to any other Type IIs restriction enzymes, and thereby provides a relatively long tag for sequencing and comparison to sequence databases. Amplicon tags are then ligated together (concatenated) and cloned for sequencing using methods known to the ordinarily skilled artisan. It some instances it may be useful to separate amplicon tags from other non-tag-containing nucleic acid fragments prior to concatenation of the amplicon tags. Various methods of separating nucleic acid molecules, which are commonly known in the art, may be used for this purpose (such as, gel separation and size exclusion column separation).

Cloned integration junction amplicons (or concatenated amplicon tags) may be sequenced in any manner known in the art. Of particular use are automated sequencing facilities, which may sequence up to several thousand integration junction amplicons (or concatenated amplicon tags) in a matter of days. For example, preparation of sequencing templates from bacterial cells may be performed robotically, for example, in a multi-well structure, such as a multi-well flow-through microcentrifuge. Mixing of samples within the rotor may be automated in a similar way, which allows all necessary protocol steps to be completed without moving the sample out of the rotor.

A number of automated sequencing methods are known in the art, including automated fluorescent dye-terminator cycle sequencing, based on the chain-termination dideoxynucleotide method. This representative method uses PCR to incorporate dideoxynucleotides, which contain fluorescent dyes, in a primer extension sequencing reaction. Each dideoxynucleotide base contains a different fluorescent dye which emits a characteristic wavelength, thus the identity of the dye corresponds to the final base on that fragment. The template of interest is amplified in the presence of appropriate primers, DNA polymerase, unlabeled dNTPs, and fluorescently labeled ddNTPs. Sequencing primers will typically be selected based on known sequencing primer binding sites in the cloning vector. Thereafter, the PCR reaction is run in a single lane on a polyacrylamide gel or microcapillary tube in an automated sequencer to separate fragments according to size. As the fragments are electrophoresed, the emission wavelength of each fragment is detected. The data is compiled into a gel image, analyzed with commercially available software and the resulting sequence is provided.

A typical sequencing reaction will most often yield sufficient information from which to identify integration junction sites, for instance by comparison to known sequence(s) in database(s).

7. Analysis of Integration Junction Sequence Data

An integrant integration site may be identified on the basis of non-integrant flanking nucleic acid sequence(s) present in integration junction amplicon sequences (or concatenated amplicon tags). Non-integrant flanking sequences may be identified in integration junction amplicon sequences (or concatenated amplicon tags) in any manner known in the art.

In one example, integration junction amplicon sequences can be analyzed for the presence of known integrant sequences. Generally, integrant-specific sequences directly segue into non-integrant flanking sequences, which marks the precise location where an integrant integrated. In another example, integration junction amplicon sequences (or concatenated amplicon tags) can be analyzed for the presence of known linker sequences. Generally, linker-specific sequences directly segue into non-integrant flanking sequences, which provides another marker of the precise location where an integrant integrated. In still another example, integration junction amplicon sequences can be analyzed for the presence of known integrant sequences and known linker sequences. Unidentified sequences located between known integrant sequences and known linker sequences likely represent non-integrant flanking sequences.

A sufficient number of consecutive nucleotides of non-integrant flanking sequence can be compared against known sequence databases (also referred to as a “reference sequence”), which correspond to the non-integrant sequences. For example, integration sites in human genomic DNA may be identified by comparison of non-integrant flanking sequences to the human genome database. In one embodiment, an integration site may be identified based on no more than about 200 base pairs of non-integrant flanking sequence. In other embodiments, an integration site may be identified based on no more than about 100 base pairs, no more than about 75 base pairs, no more than about 50 base pairs, no more than about 30 base pairs, or no more than about 20 base pairs of non-integrant flanking sequence.

The complete genomic sequences are known for humans and a variety of other organisms, including, Mus musculus, Rattus norvegicus (rat), Danio rerio (zebrafish), Avena sativa (oat), Glycine max (soybean), Hordeum vulgare (barley), Lycopersicon esculentum (tomato), Oryza sativa (rice), Triticum aestivum (bread wheat), Zea mays (corn), Arabidopsis thaliana, Caenorhabditis elegans, Drosophila melanogaster, Encephalitozoon cuniculi, Guillardia theta nucleomorph, Saccharomyces cerevisiae, Plasmodium falciparum, Schizosaccharomyces pombe, and hundreds of prokaryotic organisms.

Comparison of non-integrant flanking sequences to known reference sequences may be performed, for example, using the BLAT alignment tool (Kent, Genome Res., 12(4):656-664, 2002). In particular examples, human, non-integrant flanking sequence can be compared to the human genome using either a BLAT web batch query to the human genome browser at the University of California Santa Cruz (Kent et al., Genome Res., 12:996-1006, 2002) or through a stand alone BLAT server.

Mapped reference sequence location(s) for each non-integrant flanking sequence may be stored in a relational database. In some examples, non-integrant flanking sequences that are mapped to particular locations in the reference sequence (for example, the human genome) with greater than about 80%, about 90%, about 95% identity are selected for further analysis. The relational database may optionally contain coordinates for all RefSeq genes and other reference sequence features. All information about a specific integration and its relation to reference sequence features, such as genes, can be retrieved and categorized by querying the database.

V. Determining the Risk Potential of an Integrating Gene Therapy Vector

The disclosed methods of identifying integrant integration sites can be used to assess the risk potential of integrating gene therapy vectors. It is believed that a gene therapy vector that integrates randomly in the target nucleic acid molecule, such as a human genome, poses a relatively small risk (Kohn et al., Molecular Therapy, 8(2): 180-187, 2003). Risks associated with integration of a gene therapy vector include, for example, a preference for the vector (i) to integrate in or near actively transcribed genes, (ii) to consistently affect the activity (for example, up regulate or down regulate expression) of one or more gene(s) involved (directly or indirectly) in a vital cell process (such as, cell cycle control or cell metabolism), (iii) to inactivate tumor suppressor genes or activate oncogenic genes increasing the likelihood of the occurrence of cancer (see, for example, Shen et al., J. Virol., 77(2):1584-1588).

A method of determining the risk potential of an integrating gene therapy vector includes isolating a nucleic acid molecule having at least one integrated integrating gene therapy vector. Nucleic acid molecules useful in this method may be isolated from any biological sample, which may include integrant-containing nucleic acid molecules, using known methods (as previously described). Useful biological samples may include, for example, isolated cells, whole blood, plasma, serum, tears, bone marrow, lung lavage, mucus, saliva, urine, pleural fluid, spinal fluid, gastric fluid, sweat, semen, vaginal secretion, sputum, fluid from ulcers and/or other surface eruptions, blisters, abscesses, extracts of tissues, cells or organs, or any other type of sample that may include nucleic acids of the subject.

In some examples, one or more isolated cells, such as stem cells, are infected with an integrating gene therapy vector. Such infection may occur in a laboratory setting and, optionally, be a step in preparing the infected cells for administering to a subject as a medical treatment. In other examples, a biological sample is taken from a subject, for instance a subject who has previously received treatment with an integrating gene therapy vector or cells treated with an integrating gene therapy vector. In particular examples, a subject will have received treatment with cell (such as, stem cells) treated with an integrating gene therapy sufficiently in advance of collection of the biological sample to permit grafting and re-population of treated stem cells; for example, at least about 3 months, or at least about 6 months after the subject's treatment. In other examples, an integrating gene therapy vector (or cells treated with an integrating gene therapy vector) may be administered to a subject at least 5 days, at least 7 days, at least 14 days, or at least 21 days prior to collection of a biological sample from the subject. In specific examples, the biological sample comprises blood or bone marrow.

Integration sites of an integrating gene therapy vector may be determined and mapped in relation to at least one reference point in the nucleic acid molecule of interest, as previously described. In some examples, the risk potential of the integrating gene therapy vector is relatively high when substantial numbers of integration sites are located near actively transcribed regions of the nucleic acid molecule. In other examples, the risk potential of the integrating gene therapy vector is relatively low when the distribution of integration sites is substantially random in relation to actively transcribed regions of the nucleic acid molecule.

Based on such evaluation, a practitioner can design lower-risk vectors, redesign existing vectors, and/or counsel potential recipients.

The following examples are provided to illustrate certain particular features and/or embodiments. These examples should not be construed to limit the invention to the particular features or embodiments described.

### EXAMPLES

### Example 1

### Generation of MLV and HIV-1 Integration Site Libraries with Host Cell 3′-Flanking Sequences

This example demonstrates that MLV and HIV-1 integration site libraries consisting predominantly of host cell 3′-flanking sequences can be generated and sequenced in as little as seven days.

MLV virus pseudotyped with vesicular stomatitis virus glycoprotein G (VSV-G) was prepared as described (Chen et al., J. Virol., 76:2192-2198, 2002). 5×10⁵ HeLa cells at 25% confluence were infected with MLV virus of estimated titer of 10⁸ infection units (IU)/ml for 4 hours with 8 μg/ml of polybrene. The supernatants were removed and fresh media was added. The cells were harvested at 48 hours post infection.

pLenti6-GFP virus, a VSV-G pseudotyped HIV-1 based vector, was prepared according to the manufacturer's protocol (Invitrogen, Carlsbed, Calif.) to infect HeLa cells as described above with an estimated titer of 10⁵ IU/ml. Wild type HIV-1 virus was produced by transfection of the plasmid pNL4-3 encoding full-length infectious HIV-1 virus (Adachi et al., J. Virol., 59:284-291, 1986). H9 cells were infected with wild type HIV-1 virus transfection supernatant for 2 days, extensively washed, and harvested after an additional 2-day incubation priod.

Genomic DNA from infected cells was isolated using lysis buffer containing proteinase K and SDS (as described in Wu et al., Science, 300(5626):1749-1751, 2003). The DNA was then digested with MseI and either PstI or BglII. MseI is known to cut human genomic DNA frequently (the median length of human genomic fragments generated by MseI is about 70 bp). Amplification of shorter fragments is known to reduce PCR bias against large fragments and allow the read-through of most fragments in a single sequence pass (Cheung and Nelson, Proc. Natl. Acad. Sci. USA, 93:14676-14679, 1996). The second enzyme (either PstI or BglII) was used to prevent the amplification of an internal viral fragment from the 5′LTR. The fragments were then ligated to the MseI linker (created by annealing oligonucleotides having the sequences set forth in SEQ ID NOs: 1 and 2). Linker-mediated PCR (LM-PCR) was performed with one primer specific to the LTR (SEQ ID NO: 5 for MLV and SEQ ID NO: 7 for HIV-1) and the other primer to the linker (SEQ ID NO: 3 for both MLV and HIV-1) with the following conditions: pre-incubation at 95° C. for 2 min, then 25 cycles of 95° C. for 15 sec, 55° C. for 30 sec and 72° C. for 1 min.

The PCR products were diluted 1:50 and nested PCR was performed under the same conditions using a second set of primers, one bound to the LTR (SEQ ID NO: 6 for MLV and SEQ ID NO: 8 for HIV-1) and the other bound to the linker (SEQ ID NO: 4 for both MLV and HIV-1). Nested PCR products (predominantly representing host cell 3′ genomic flanking sequences) were directly shotgun cloned without purification into the TOPO TA cloning kit (Invitrogen, Carlsbed, Calif.) following the manufacturer's instructions, and then transformed into One Shot® TOP10 (Invitrogen) competent cells to form libraries of integration junction fragments.

The sequencing of the library was carried out by the fully automated NIH Intramural Sequencing Center. The number of colonies per milliliter for the library was determined. Then, the library was plated on LB agar plates at the appropriate density for automated picking. Individual colonies were picked with a robot colony picker. Plasmid preparation and sequencing was fully automated using a 384-well format.

Generation of MLV and HIV-1 integration site libraries and sequencing of the inserts as described in this example was completed in 7 days. Once genomic DNA containing viral integrations is available, as little as 5 days may be needed to obtain sequence information; for example, construction of a typical integration junction fragment library may be completed in no more than 2 days, and sequencing can be completed in about 3 days if a commercial sequence provider is used. In comparison, a method such as described in Schroder et al. (Cell, 110:521-529, 2002), which digests the genomic DNA into much longer fragments and requires a gel purification step (thereby introducing amplification and cloning biases), can take months.

Oligonucleotides used in this example are listed in Table 2.

TABLE 2

| | |
|---|---|
| Name | Sequence (shown 5′ to 3′) |

| | |
|---|---|
| MseI linker+ | GTAATACGACTCACTATAGGGCTCCGCTTAAGGGAC |
| | (SEQ ID NO: 1) |
| MseI linker− | PO₄-TAGTCCCTTAAGCGGAG-NH₂ |
| | (SEQ ID NO: 2) |
| MLV 3′LTR | GACTTGTGGTCTCGCTGTTCCTTGG |
| primer | (SEQ ID NO: 5) |
| MLV 3′LTR | GGTCTCCTCTGAGTGATTGACTACC |
| nested primer | (SEQ ID NO: 6) |
| HIV-1 3′LTR | AGTGCTTCAAGTAGTGTGTGCC |
| primer | (SEQ ID NO: 7) |
| HIV-1 3′LTR | GTCTGTTGTGTGACTCTGGTAAC |
| nested primer | (SEQ ID NO: 8) |
| linker primer | GTAATACGACTCACTATAGGGC |
| | (SEQ ID NO: 3) |
| linker nested | AGGGCTCCGCTTAAGGGAC |
| primer | (SEQ ID NO: 4) |

### Example 2

### Mapping and Analysis of MLV and HIV-1 Integration Sites

This example demonstrates that substantial numbers of HIV-1 and MLV integration sites can be accurately mapped to the human genome from sequence data collected as described in Example 1. Mapping results demonstrate that MLV has a preference for integration in the region surrounding the transcriptional start sites in the human genome, while HIV-1 prefers to integrate in the transcribed region of human genes.

The BLAT program (Kent, Genome Res., 12(4):656-664, 2002) was used to map sequences generated in Example 1 to the human genome as provided in the University of California Santa Cruz (UCSC) Human Genome Project Working Draft, November 2002 freeze (Karolchik et al., Nucl. Acids Res., 31:51-54, 2003). All analysis used the annotation database specific to that build. A sequence was only considered to be from a genuine integration event if it (1) contained both the 3′LTR sequence from the nested primer to the end of 3′LTR (CA) and the linker sequence, (2) matched to a genomic location starting immediately (within 3 bases) after the end of 3′LTR (which was marked by the base sequence “CA”), (3) showed 95% or greater identity to the genomic sequence over the high quality sequence region, and (4) matched to no more than one genomic locus with 95% or greater identity.

2304 clones from the MLV HeLa integration library were sequenced. 1379 of these clones had both 3′LTR and linker sequence. The median length of inserts with both LTR and linker sequence was 78 bps. 903 sequences met all of the above criteria and could be mapped to a unique genomic locus. The remaining sequences were either too short to map to any location, were duplicate clones, or mapped to multiple locations. Only 16 integration sites were sequenced in more than one clone and none appeared more than twice, suggesting that saturation of the integration site library was not reached.

244 integrations from the wild type HIV-1 virus infected human H9 cell line and 135 integrations from the pseudotyped HIV-1 vector virus infected human HeLa cell line were mapped for a total of 379 integrations.

1. Data Analysis

The coordinates of RefSeq genes, CpG islands and other annotation tables for the November 2002 human genome freeze were downloaded from the UCSC genome project website. An integration was deemed to have “landed” in a gene only if it the integration was between the transcriptional start and transcriptional stop boundaries of one of the 18,214 RefSeq genes mapped to the human genome. RefSeq genes are curated based on known mRNA transcripts and do not rely on gene prediction programs, thus avoiding potential computational bias. Integrations were also analyzed in various sized windows around transcriptional start sites, transcription end sites, and CpG islands. To analyze the distribution of integrations within genes, RefSeq genes were arbitrarily divided into 8 equal fragments from 5′ end of transcripts to 3′ end of transcripts. The distribution of MLV and HIV-1 integration sites were compared to each other and to a set of 10,000 random-integration coordinates generated by computer.

The analysis revealed that 62% (152/244) of HIV-1 integrations in H9 cells landed in RefSeq genes and 50% (67/135) of pseudotyped HIV-1 integrations in HeLa cells landed in RefSeq genes. Since there was no statistically significant difference between the two HIV-1 datasets, they were combined to show that 58% of the HIV-1 integrations into the human genome landed in RefSeq genes. For the MLV integrations, 34% of the integrations (309/903) landed in RefSeq genes. In contrast, only 22.4% of a set of 10,000 computer simulated random integrations landed in RefSeq genes, which was significantly fewer than for both HIV-1 and MLV (Chi-square test, p<0.0001).

It was next determined whether the promoter regions of genes were favored target sites for MLV and/or HIV-1 integration. Since no accurate coordinates for the promoter regions of RefSeq genes are available, integrations were analyzed in terms of various window sizes on either side of the +1 start site for RefSeq genes.

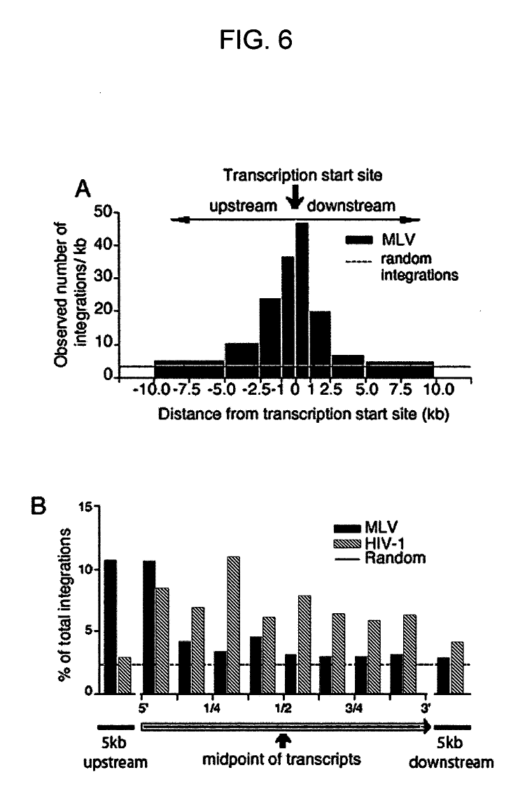

As shown in FIG. 6A, the smaller the window size surrounding the transcriptional start site, the higher the density of observed MLV integrations. The number becomes too small to draw statistically valid conclusions when the window size is smaller than 1 kb. In contrast, the percentage of HIV-1 integration sites that landed in the 5 kb upstream regions of RefSeq genes is statistically indistinguishable from random placements (see FIG. 6B).

MLV integrations were found to be distributed evenly upstream or downstream of the transcriptional start site (FIG. 6A). This is very different from HIV-1 integrations, which highly favor the entire length of the transcriptional regions, but not the regions upstream of the transcriptional start (FIG. 6B). No preferences was observed for the regions just downstream of the RefSeq transcripts for either MLV or HIV-1 integrations (FIG. 6B).

CpG islands are thought to be commonly associated with the transcriptional start sites in the vertebrate genome (Bird, Nature, 321:209-213, 1986; Larsen et al., Genomics, 13:1095-1107, 1992). Thus, the association between MLV and HIV-1 integration sites and documented human CpG islands (see, UCSC human genome November 2002 freeze) was determined. 16.8% (152/903) of the MLV integrations landed in the region 1 kb+/− of the 27,704 documented human CpG islands, which is 8 times higher than the value of 2.1% for random integrations. However, only 2.1% of HIV-1 integrations landed in the region 1 kb+/− of the same CpG islands.

Table 3 summarizes the results described in this example.

TABLE 3
MLV and HIV-1 integration site distribution.

| | Percentage of integrations | | |
|---|---|---|---|
| | MLV | HIV-1^{‡} | Random^{§} |
| Within RefSeq Genes | 34.2*^{†} | 57.8* | 22.4 |
| Within 5 kb upstream of genes | 11.2*^{†} | 2.9 | 2.1 |
| Within 5 kb downstream of genes | 3.4 | 4.5 | 2.1 |
| Within 5 kb +/− transcription start sites | 20.2*^{†} | 10.8* | 4.3 |
| Within 1 kb +/− CpG islands | 16.8*^{†} | 2.1 | 2.1 |

The total number of mapped integrations were 903 and 379 for MLV and HIV-1, respectively.
*p < 0.0001 compared to random integration using a Chi-square test.
^{†}p < 0.0001 compared to HIV-1 integration using a Chi-square test.
^{‡}Pooled integration data from pseudotyped and infectious HIV-1.
^{§}From a set of 10,000 computer simulated random integrations.

2. MLV Integration Targets Transcriptionally Active Genes

To determine if MLV-targeted genes are transcriptionally active in HeLa cells, the publicly available Gene Expression Omnibus (GEO) database (Edgar et al., Nuc. Acids Res., 30:207-210, 2002) was used. Two independent sets of microarray data based on HeLa cell mRNA were analyzed (GSM2145, GSM2177).

Of the 196 MLV integrations that were within 5 kb+/− of transcription start sites of RefSeq genes, 79 were represented on the arrays. The median expression level for these 79 genes was approximately 1.8 fold higher than that of all the genes on the arrays (1911/1288 in GSM2145 and 1052/487 in GSM2177; Mann-Whitney test, p<0.0001). More than 75% of the 79 genes were expressed at levels above the median level of all genes. The mean expression level for these 79 genes is also higher than that of all genes on the arrays (2289/1648 in GSM2145 and 1328/863 in GSM2177). Since the expression levels of genes on the array do not follow a normal distribution, the non-parametric Mann-Whitney test was used to compare the median of the 79 genes to the median for all genes on the array (p<0.0001).

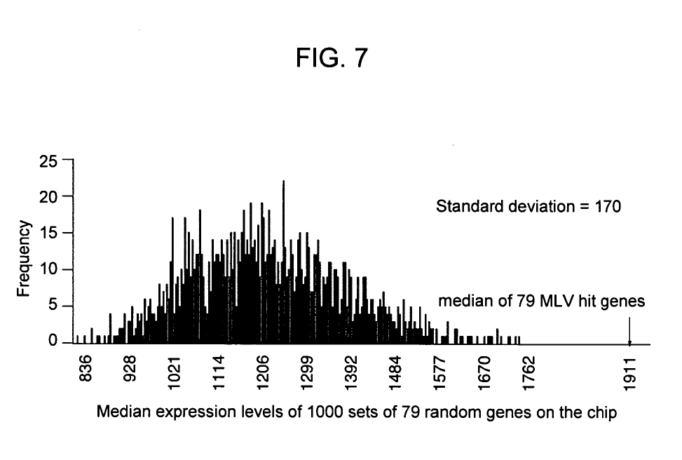

The median expression level of the 79 genes represented on the arrays was also compared to that value of 1000 sets of 79 genes randomly picked by computer. As shown in FIG. 7, the median expression level of the 79 hit genes falls outside 4 standard deviations of the mean of 1000 sets of randomly picked genes.

The different integration profiles for MLV and HIV-1 indicate that there are fundamental mechanistic differences influencing site preferences for the two viruses. It also suggests the risk factors for the use of MLV- or HIV-1-based vectors for gene therapy will not be identical. These differences underscore the usefulness of the disclosed methods of rapidly mapping viral integrations sites. Such methods may be used to characterize the integration preferences of different retroviral gene therapy systems so as to fully understand the risks and advantages of such systems.

### Example 3

### No Detectable Bias is Introduced by Mapping Methods

This example demonstrates that that the MLV and HIV-1 integrations identified in Example 1 were not biased by the in vitro amplification technique used to isolate them.

One concern in cloning and mapping of a large number of retroviral integration sites to the genome using conventional PCR and computational methods, is that biases to the data can be introduced. In contrast, no detectable bias was introduced using the methods disclosed herein.

PCR is known to work more efficiently on shorter templates in a mixed population of templates. The key to avoiding amplification bias is to generate short, similar sized fragments (see, for example, Cheung and Nelson, Proc. Natl. Acad. Sci. USA, 93:14676-14679, 1996). Because of the availability of essentially the entire human genome sequence, computational restriction enzyme digestions were performed with several candidate enzymes, including MseI, Rsa I, and Taq I. MseI (having the recognition site, T|TAA) was chosen as a useful enzyme because it generates very short genomic DNA fragments (with a median length of 70 bp, and 95% fragments are less than 500 bp).

To determine if the choice of MseI introduced a bias toward AT rich regions, the GC content in various window sizes surrounding all the mapped integration sites was analyzed. As shown in Table 4, the GC content of regions near MLV integration sites was not statistically different than the genome-wide average value. If it shows any bias, Table 4 shows a small bias for GC rich regions, apparently reflecting the fact that MLV integration favors the regions around CpG islands (as discussed in Example 2).

TABLE 4
GC content around mapped MLV integration sites, transcriptional
start sites comparing to the whole genome

| Window sizes around all | |
|---|---|
| MLV integration sites | GC content (%) |
| 50 bp | 42 |
| 100 bp | 42 |
| 250 bp | 43 |
| 500 bp | 44 |
| 1000 bp | 44 |
| Transcriptional start sites +/−10 kb | 46 |
| Genome-wide average | 41 |

It is believed that the methods described in Example 1 did not introduce genomic regional bias because the same method was used to clone and map integration sites for two different retroviruses, and the results showed that HIV-1 and MLV have different integration profiles.

### Example 4

### Amplification of 3′ and 5′ Integration Junction Fragments

This example demonstrates that non-integrant flanking sequences on one or both sides of an integrant (that is, both upstream (5′) and/or downstream (3′)) can be amplified.

pGT is a plasmid that contains a single MLV retroviral genome (Naviaux et al., J. Virol., 70(8):5701-5705, 1996). GT186 is a cell line, the genome of which contains three known integrations of a MLV-based retroviral genome and a separate locus that expresses the MLV gag-pol polypeptide for viral packaging (Chen et al., J. Virol., 76(5):2192-2198, 2002). The MLV-based retroviral genome in GT186 contains only DNA (RNA) sequences necessary for integration, and the separate locus provides all the retroviral proteins necessary for integration; thus, the retroviruses that are packaged into infectious particles are unable to replicate once infection has taken place. Gene therapy treatments commonly use retroviral vectors modified in the manner of the GT186 MLV-based retroviral genome. The pGT integrant and the GT186 integrants may be referred to in this example as “MLV integration(s)” or “MLV integrant(s).”

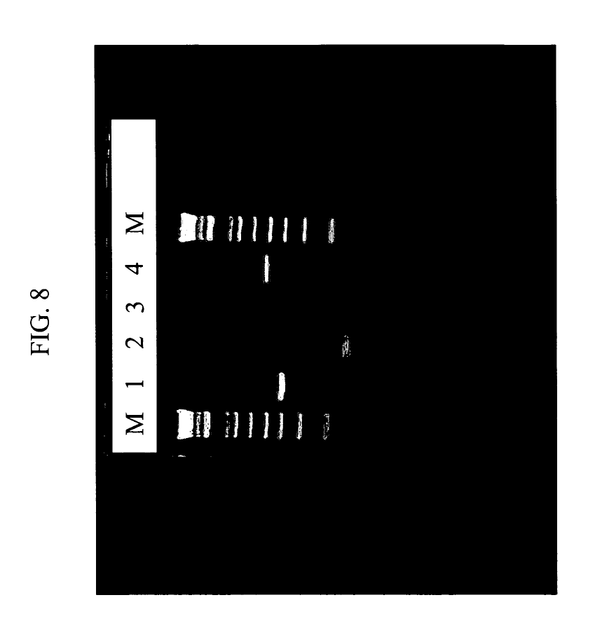

Integration junction fragments containing the 3′ end of the MLV integrant(s) were obtained from both pGT plasmid DNA and GT 186 genomic DNA by linker-mediated amplification as described in Example 1. FIG. 8, lane 1 shows a single integration junction fragment (approximately 400 base pairs) representative of a single MLV integration in pGT. FIG. lane 3 shows three integration junction fragments (approximately 110, 180, and 240 base pairs) representative of the three MLV integrations in GT186 genomic DNA. The estimated sizes of the fragments on the gel are consistent with the expected sizes of the 3′ integration junction fragments for the respective MLV integrant(s).

Integration junction fragments containing the 5′ end of the MLV integrant(s) were obtained essentially as described in Example 1, except (i) EcoRI was used in place of PstI as the N2 restriction enzyme, and (ii) the following MLV 5′ terminal-repeat-specific primers (TRPs) were used instead of “MLV 3“LTR primer” and “MLV 3” LTR nested primer” (each of which are shown in Table 2):

| | |
|---|---|
| Name | Sequence (shown 5′ to 3′) |

| | |
|---|---|
| MLV 5′LTR primer | TAGCTTGCCAAACCTACAGGT |
| | (SEQ ID NO: 13) |
| MLV 5′LTR nested | ACCTACAGGTGGGGTCTTTCA |
| primer | (SEQ ID NO: 14) |

FIG. 8, lane 2 shows a single integration junction fragment (approximately 150 base pairs) representative of a single MLV integration in pGT. FIG. lane 4 shows three integration junction fragments (approximately 150, 400, and 520 base pairs) representative of the three MLV integrations in GT186 genomic DNA. The estimated sizes of the fragments on the gel are consistent with the expected sizes of the 5′ integration junction fragments for the respective MLV integrant(s).

### Example 5

### Amplification of 3′ and 5′ Integration Junction Fragments from Varying Amounts of Target DNA

This example demonstrates that at least as little as 5 ng of genomic DNA can be successfully used to produce either 5′ or 3′ integration junction fragments using the disclosed methods.

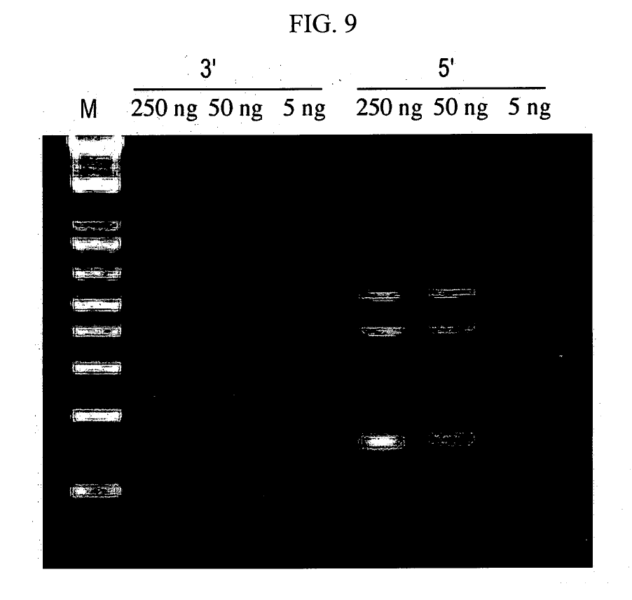

5′ and 3′ integration junction fragments were amplified, as described in Example 4, from varying amounts of GT186 genomic DNA. As shown in FIG. 9, three integration junction fragments (corresponding to the three MLV integrations in GT186 genomic DNA) were amplified in each case. The sizes of the fragments correspond to the expected sizes of the respective 5′ and 3′ integration junction fragments as described in Example 4.

FIG. 9 shows that the expected integration junction fragments were obtained over a 50-fold range of genomic DNA starting material. These results demonstrate the sensitivity of the disclosed methods; for example, 5′ and 3′ integration junction fragments may be produced from as little as 5 ng of genomic DNA.

### Example 6

### Amplification of Integration Junction Fragments Using RsaI

This example demonstrates that integration junction fragments can be amplified with various restriction enzymes.

5′ and 3′ integration junction fragments were amplified from 5 ng of pGT plasmid and 5 ng of GT 186 genomic DNA, as described in Example 4, except RsaI was substituted for MseI in the restriction enzyme digestion. As a result of the restriction enzyme substitution, an extension-dependent linker having an RsaI-compatible end was used, and primary and nested primers specific for this linker were designed. The oligonucleotides used for the RsaI-specific linker and the linker primers are shown below:

| | |
|---|---|
| Name | Sequence (shown 5′ to 3′) |

| | |
|---|---|
| RsaI | GTAATACGACTCACTATAGGGCACGCGTGGTCCATGGG |
| linker+ | (SEQ ID NO: 9) |
| RsaI | PO₄-CCCATGGACCAC-NH₂ |
| linker− | (SEQ ID NO: 10) |
| RsaI linker | GTAATACGACTCACTATAGGGC |
| primer | (SEQ ID NO: 11) |
| RsaI linker | ACTATAGGGCACGCGTGGT |
| nested | (SEQ ID NO: 12) |
| primer | |

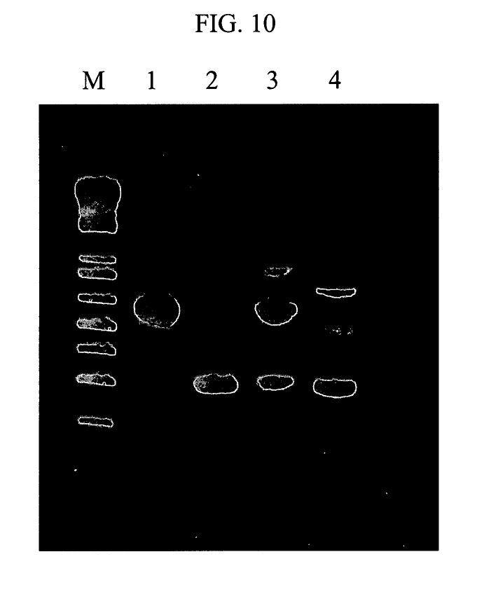

As shown in FIG. 10, a single 5′ integration junction fragment (lane 1) and a single 3′ integration junction fragment (lane 2) were amplified from RsaI/EcoRI— and RsaI/PstI-digested pGT plasmid DNA, respectively. These fragments include the 5′ end and the 3′ end, respectively, of the single MLV genome present in pGT. As further shown in FIG. 10, three 5′ integration junction fragments (lane 3) and three 3′ integration junction fragments (lane 4) were amplified from RsaI/EcoRI— and RsaI/PstI-digested GT186 genomic DNA, respectively. These fragments correspond to the 5′ ends and the 3′ ends, respectively, of the three MLV integrations present in GT186 genomic DNA.

While this disclosure has been described with an emphasis upon particular embodiments, it will be apparent to those of ordinary skill in the art that variations of the particular embodiments may be used and it is intended that the disclosure may be practiced otherwise than as specifically described herein. Accordingly, this disclosure includes all modifications encompassed within the spirit and scope of the disclosure as defined by the following claims:

TABLE 1
Restriction Enzymes Having Recognition Sites of Five or Fewer Base Pairs

| | Recognition | | Recognition | | Recognition |
|---|---|---|---|---|---|
| Enzymes | Sequence | Enzymes | Sequence | Enzymes | Sequence |
| AcaIV | GGGC | BamNxI | G⇓GWCC | BpuSI | GGGAC |
| AccII | CG⇓CG | BanAI | GG⇓CG | BsaCI | CCNGG |
| Acc38I | CCWGG | BavAII | G⇓GNCC | BsaLI | AGCT |
| AceI | G⇓CWGC | BavBII | G⇓GNCC | BsaNI | CCWGG |
| AciI | CCGC | BbvI | GCAGG | BsaPI | GATC |
| AclWI | GGATC | BcaI | GCGC | BsaRI | GGCC |
| AcuII | CCWGG | BccI | CCATC | BsaSI | GGNCC |
| AeuI | CC⇓WGG | Bce22I | G⇓GNCC | BsaUI | GCAGC |
| AfaI | GT⇓AG | Bce7II | GGCC | BsaZI | CCGG |
| AfII | G⇓GWCC | Bce243I | ⇓GATC | BscAI | GCATC |
| Afl83II | GGCC | Bce31293I | CGCG | BscFI | ⇓GATC |
| AglI | CC⇓WGG | BceAI | ACGGC | BscGI | CCCGT |
| AhaI | CC⇓SGG | BceBI | CG⇓CG | BscHI | ACTGG |
| AhaB1I | GGNCC | BceRI | CGCG | BscPI | CTNAG |
| AjnI | ⇓CCWGG | BcefI | ACGGC | BscQI | GGGC |
| AluI | AG⇓CT | BchI | GCAGC | BscQII | GTCTC |
| AlwI | GGATC | BciBII | CC⇓WGG | BscUI | GCATC |
| Alw26I | GTGTC | BcnI | CC⇓SGG | BscWI | GGGAC |
| AlwXI | GGAGC | Bco27I | C⇓CGG | BseI | GGCC |
| AorI | CC⇓WGG | Bco33I | GGCC | BseII | ACTGG |
| ApaORI | CC⇓WGG | BctI | ACGGC | Bse9I | GGCC |
| ApeKI | GCWGC | BcuAI | G⇓GWCC | Bse16I | CC⇓WGG |
| ApuI | GGNCC | BecAII | GG⇓CC | Bse17I | CC⇓WGG |
| ApyI | CCSWGG | BepI | CG⇓CG | Bse24I | CC⇓WGG |
| AseII | CC⇓SGG | BfaI | C⇓TAG | Bse54I | GGNGC |
| AspII | CCSGG | Bfi57I | ⇓GATC | Bse126I | GGCC |
| Asp697I | GGWCC | Bfi105I | GGNCC | BseBI | CCSWGG |
| Asp742I | GGCC | Bfi458I | GGCC | BseGI | GGATG |
| Asp748I | CCGG | BfuCI | ⇓GATC | BseKI | GCAGC |
| AspBII | GGWCC | BhaI | GCATC | BseMII | CTCAG |
| AspCNI | GCCGC | BhaII | GGCC | BseNII | ACTGG |
| AspDII | GGWCC | Bim19II | GG⇓CC | BseQI | GG⇓CC |
| Asp2HI | CCWGG | BinI | GGATC | BseXI | GCAGC |
| Asp16HI | GTAC | BinSI | CCWGG | BshI | GG⇓CC |
| Asp17HI | GTAC | BliI | GGCG | Bsh1236I | CGCG |
| Asp18HI | GTAC | BloNORF564P | GATC | BshAI | GGCC |
| Asp29HI | GTAG | BloNORF1473P | CGWGG | BshBI | GGCG |
| AspLEI | GCG⇓C | BlopNAC1P | CCWGG | BshCI | GGCC |
| AspMDI | ⇓GATC | BluII | GGCC | BsbDI | GGCC |
| AspS9I | G⇓GNCC | Bme12I | ⇓GATC | BshEI | GGCC |
| AspTIII | GGCC | Bme18I | G⇓GWCC | BshFI | GG⇓CG |
| AsuI | G⇓GNCC | Bme46I | GGCC | BshGI | CC⇓WGG |
| AsuC2I | CC⇓SGG | Bme74I | GGCC | BshKI | G⇓GNCC |
| AsuHPI | GGTGA | Bme216I | G⇓GWCC | BshMI | CCGG |
| AsuMBI | GATC | Bme361I | GG⇓CC | BsiAI | GGCC |
| AtuII | CCWGG | Bme585I | CCCGC | BsiDI | GGCG |
| AtuII | CCWGG | Bme1390I | CC⇓NGG | BsiHI | GGCC |
| AtuBI | CCWGG | Bme2095I | CCWGG | BsiLI | CC⇓WGG |
| AvaII | G⇓GWCC | Bme2494I | GATC | BsiSI | C⇓CGG |
| AvcI | G⇓GNCC | BpsI | GGNCC | BsiUI | CCWGG |
| AvrBI | GGGG | Bpu95I | CG⇓CG | BsiVI | CCWGG |
| Bac36I | G⇓GNCC | Bpu1811I | GCNGC | BsiZI | G⇓GNCC |
| Ba1228I | G⇓GNCC | BpuFI | GGATC | BsmAI | GTCTC |
| Ba1475I | GGCC | BpuJI | CCCGT | BsmEI | GAGTC |
| Ba13006I | GGCC | BpuNI | GGGAC | BsmFI | GGGAC |
| BsmNI | GCATC | Bsp143I | ⇓ATC | BssCI | GGCC |
| BsmXII | GATC | Bsp147I | GATC | BssFI | GCNGC |
| BsoI | CGNGG | Bsp211I | GG⇓CC | BssGII | GATC |
| BsoFI | GC⇓NGC | Bsp226I | GGCC | BssIMI | GGGTC |
| BsoGI | CCWGG | Bsp317I | CCWGG | BssKI | ⇓CCNGG |
| BsoHI | ACTGG | Bsp423I | GGAGC | BssXI | GCNGC |
| BsoMAI | GTCTC | Bsp548I | CCNGG | Bst1I | CC⇓WGG |
| BspI | GATC | Bsp881I | GGCG | Bst2I | CC⇓WGG |
| Bsp5I | CCGG | Bsp1260I | GGWCG | Bst11I | ACTGG |
| Bsp6I | GC⇓NGC | Bsp1261I | GGCG | Bst12I | GCAGC |
| Bsp7I | GCSGG | Bsp1591II | CCGG | Bst19I | GCATC |
| Bsp8I | CCSGG | Bsp1593I | GGCC | Bst19II | ⇓GATC |
| Bsp9I | GATC | Bsp1894I | G⇓GNCC | Bst38I | CC⇓WGG |
| Bsp18I | GATC | Bsp2013I | GGCC | Bst40I | G⇓CGG |
| Bsp23I | GGCC | Bsp2095I | ⇓GATC | Bst71I | GCAGC |
| Bsp44I | CCWGG | Bsp2362I | GGCG | Bst100I | CC⇓WGG |
| Bsp44II | GGGC | Bsp2500I | GGCG | Bst295I | GTNAG |
| Bsp47I | CCGG | BspAI | ⇓GATC | Bst1274I | GATC |
| Bsp48I | CCGG | BspANI | GG⇓CC | BstCI | GGCC |
| Bsp49I | GATC | BspBII | G⇓GNCC | Bst4GI | ACN⇓GT |
| Bsp50I | CG⇓CG | BspBDG2I | GGCC | BstDEI | C⇓TNAG |
| Bsp51I | GATC | BspBRI | GG⇓CC | BstDZ247I | CCCGT |
| Bsp52I | GATC | BspBSE18I | GGCC | BstEIII | GATC |
| Bsp53I | CCNGG | BspBake1I | GGCC | BstENII | ⇓GATC |
| Bsp54I | GATC | BspCHE15I | GGCC | BstF5I | GGATG |
| Bsp55I | CCSGG | BspCNI | CTCAG | BstFNI | CG⇓CG |
| Bsp56I | CCWGG | BspFI | ⇓GATC | BstFZ438I | CCCGC |
| Bsp57I | GATC | BspF4I | G⇓GNCC | BstGII | CCWGG |
| Bsp58I | GATC | BspF53I | GGWCC | BstH9I | GGATC |
| Bsp59I | GATC | BspF105I | CCSGG | BstHHI | GCG⇓C |
| Bsp60I | GATC | BspGHA1I | GGCC | BstJI | GGCC |
| Bsp61I | GATC | BspH43I | CCWGG | BstJZ301I | C⇓TNAG |
| Bsp64I | GATC | BspH106II GGCC | BstKTI | GAT⇓C | |
| Bsp65I | GATC | BspJI | ⇓GATC | BstM6I | CC⇓WGG |
| Bsp66I | GATC | BspJ64I | GATC | BstMZ611I | ⇓CCNGG |
| Bsp67I | ⇓ATC | BspJ67I | CCSGG | BstNI | CC⇓WGG |
| Bsp70I | CGCG | BspJ76I | CGCG | BstOI | CC⇓WGG |
| Bsp71I | GGWCC | BspJ10SI | GGWCC | BstOZ616I | GGGAC |
| Bsp72I | GATC | BspKI | GG⇓CC | BstPZ418I | GGATG |
| Bsp73I | CCNGG | BspKT6I | GAT⇓C | Bst4QI | GGWCG |
| Bsp74I | GATG | BspLAI | GCG⇓C | Bst7QII | CGWGG |
| Bsp76I | GATC | BspLRI | GGCC | BstSCI | ⇓CCNGG |
| Bsp91I | GATG | BspLU11III | GGGAC | Bst31TI | GGATC |
| Bsp100I | GGWCC | BspNI | CC⇓WGG | BstUI | CG⇓CG |
| Bsp103I | CCWGG | BspNCI | CCAGA | Bst2UI | CC⇓WGG |
| Bsp105I | ⇓GATC | BspPI | GGATC | BstV1I | GCAGC |
| Bsp116I | CCGG | BspRI | GG⇓CC | BstXII | GATC |
| Bsp122I | GATC | BspSI | CCWGG | Bsu54I | G⇓GNCC |
| Bsp123I | CG⇓CG | BspST5I | GCATC | Bsu1076I | GGCG |
| Bsp128I | GGWCC | BsrI | ACTGG | Bsu1114I | GGCG |
| Bsp132I | GGWCC | BsrAI | G⇓GWCC | Bsu1192I | CCGG |
| Bsp133I | GGWCC | BsrMI | GATC | Bsu1192II | CGCG |
| Bsp135I | GATC | BsrPII | GATC | Bsu1193I | CGCG |
| Bsp136I | GATC | BsrSI | ACTGG | Bsu1532I | CG⇓CG |
| Bsp137I | GGCC | BsrVI | GCAGC | Bsu5044I | GGNCC |
| Bsp138I | GATC | BsrWI | GGATC | Bsu6633I | CGCG |
| BsuEII | GGCG | Cfr5I | CCWGG | CviBI | G⇓ANTC |
| BsuFI | C⇓CGG | Cfr8I | GGNCC | CviCI | GANTC |
| BsuRI | GG⇓CC | Cfr11I | CCWGG | CviDI | GANTC |
| BtcI | GATC | Cfr13I | G⇓GNCC | CviEI | GANTC |
| BteI | GG⇓CC | Cfr20I | CCWGG | CviFI | GANTC |
| BthII | GGATC | Cfr22I | CCWGG | CviGI | GANTC |
| Bth84I | GATC | Cfr23I | GGNCC | CviHI | GATC |
| Bth211I | GATC | Cfr24I | CCWGG | CviJI | RG⇓CY |
| Bth213I | GATC | Cfr25I | CCWGG | CviKI | RGCY |
| Bth221I | GATC | Cfr27I | CCWGG | CviLI | RGCY |
| Bth617I | GGATC | Cfr28I | CCWGG | CviMI | RGCY |
| Bth945I | GATC | Cfr29I | CCWGG | CviNI | RGCY |
| Bth1140I | GATC | Cfr30I | CCWGG | CviOI | RGCY |
| Bth1141I | GATC | Cfr31I | CCWGG | CviQI | G⇓TAC |
| Bth1786I | GATC | Cfr33I | GGNCC | CviRI | TG⇓CA |
| Bth1997I | GATC | Cfr35I | CCWGG | CviRII | G⇓TAC |
| BtbAI | G⇓GWCC | Cfr45I | GGNCC | CviSIII | TCGA |
| BthCI | GCNG⇓C | Cfr46I | GGNCC | CviTI | RG⇓CY |
| BthCan1 | GATC | Cfr47I | GGNCC | DdeI | C⇓TNAG |
| BtbDI | CC⇓WGG | Cfr52I | GGNCC | DpnI | GA⇓TC |
| BthEI | CC⇓WGG | Cfr54I | GGNCC | DpnII | ⇓GATC |
| BtiI | GGWCC | Cfr58I | CCWGG | DsaII | GG5CC |
| BtkI | CG⇓CG | CfrNI | GGNCC | DsaIV | G⇓GWCC |
| BtkII | SGATC | CfrS37I | CCWGG | DsaV | ⇓CCNGG |
| BtsPI | GGGTC | CfuI | GA⇓TC | EacI | GGATC |
| Btu33I | GATC | Cg1I | GC⇓GC | EagKI | CCWGG |
| Btu34I | GATC | ChaI | GATC⇓ | EagMI | G⇓GWCC |
| Btu36I | GATC | Cm1467I | GATC | EcaII | CCWGG |
| Btu37I | GATC | CjeP338I | GATC | EciDI | CCSGG |
| Btu39I | GATC | CjeP338II | GCATC | Ec1II | CCWGG |
| Btu41I | GATC | CliI | GGWCC | Ecl66I | CCWGG |
| CacI | ⇓GATC | ClmI | GGCC | Ecl136I | CCWGG |
| Cac824I | GCNGC | CltI | GG⇓CC | Ecl137II | CCWGG |
| CauI | G⇓G WCC | CpaI | GATC | EclS39I | CCWGG |
| CauII | CC⇓SGG | Cpa1150I | CGCG | Ecl18kI | ⇓CCNGG |
| CboI | C⇓CGG | CpaAI | CGCG | Ec137kII | CCWGG |
| CbrI | CC⇓WGG | CpfI | ⇓GATC | Ec154kI | CCWGG |
| CceI | CCGG | CpfAI | GATC | Ec157kI | CCWGG |
| CcoP31I | GATC | Csp2I | GGCC | Ecl1zII | CCWGG |
| CcoP73I | GTAC | Csp5I | GATC | Eco38I | CCWGG |
| CcoP76I | GATC | Csp6I | G⇓TAC | Eco39I | GGNCC |
| CcoP84I | GATC | Csp1470I | GCGC | Eco40I | CCWGG |
| CcoP95I | GCGC | Csp68KI | G⇓GWCC | Eco41I | CCWGG |
| CcoP95II | GATC | Csp68KVI | CG⇓CG | Eco43I | CCNGG |
| CcoP215I | GCNGG | CspKVI | CG⇓CG | Eco47II | GGNCC |
| CcoP216I | GCNGC | Cte1179I | GATC | Eco51II | CCNGG |
| CcoP219I | GATC | Cte1180I | GATC | Eco60I | CCWGG |
| CcuI | G⇓GNCC | CteEORF387P | GATC | Eco61I | CCWGG |
| CcyI | ⇓GATC | CteTORF2122P | CCWGG | Eco67I | CCWGG |
| CdiI | CATCG | CthII | CC⇓WGG | Eco70I | CCWGG |
| Cdi27I | CCWGG | CthORFS26P | GGCC | Eco71I | GCWGG |
| CdiAI | GGNCC | CthORFS34P | GATC | Eco80I | CCNGG |
| CdiCD6I | GGNCC | CthORFS93P | GATC | Eco85I | CCNGG |
| CdiCD6II | GATC | CtyI | GATC | Eco93I | CCNGG |
| CfoI | GCG⇓C | CviAI | ⇓GATC | Eco121I | CCSGG |
| Cfr4I | GGNCC | CviAII | C⇓ATG | Eco128I | CCWGG |
| Eco153I | CCNGG | FspMI | CGCG | Hpy991XP | GANTC |
| Eco170I | CCWGG | FspMSI | G⇓GWCC | Hpy99XIP | ACGT |
| Eco179I | CCSGG | EssI | G⇓GWCC | Hpy128P | CATG |
| Eco190I | CCSGG | GmeORFC6P | GGATC | Hpy166I | TCNGA |
| Eco193I | CCWGG | GseI | GGNCC | Hpy166III | GCTC |
| Eco196II | GGNCC | GspAI | GGWCC | Hpy166IVP | CATG |
| Eco200I | CCNGG | HacI | ⇓GATC | Hpy178II | GAAGA |
| Eco201I | GGNCC | HaeIII | GG⇓CC | Hpy178VI | GGATG |
| Eco206I | CCWGG | HapII | C⇓CGG | Hpy178VII | GGCC |
| Eco207I | CCWGG | HgaI | GACGC | Hpy8829P | GATC |
| Eco254I | CCWGG | HgiBI | G⇓GWCC | Hpy85369P | CATG |
| Eco256I | CCWGG | HgiCII | G⇓GWCC | Hpy85371P | CATG |
| Eco1831I | ⇓CCSGG | HgiEI | G⇓GWCC | Hpy85372P | CATG |
| EcoHI | ⇓CCSGG | HgiHIII | G⇓GWCC | Hpy85373P | CATG |
| EcoRII | ⇓GCWGG | HgiJI | G⇓OWCC | Hpy85374P | CATG |
| Eco13kI | ⇓CCNGG | HgiS21I | GCSGG | Hpy85375P | CATG |
| Eco21kI | ⇓CCNGG | HgiS22I | CC⇓SGG | Hpy85376P | CATG |
| Eco137kI | ⇓CCNGG | HhaI | GCGC | Hpy85377P | CATG |
| EcopHSHP | CCWGG | HhaII | G⇓ANTC | Hpy85378P | CATG |
| EcopHSH2P | CCWGG | HhdI | CCWGG | Hpy85379P | GATG |
| ErpI | G⇓GWCC | HheORF238P | GATATC | Hpy85393P | CATG |
| EsaBC3I | TC⇓GA | HheORF1050P | CATG | Hpy85394P | GATG |
| EsaBC4I | GG⇓CC | HhgI | GGCC | Hpy85395P | CATG |
| EsaDix6IP | TCGA | Hin1II | CATG⇓ | Hpy85396P | CATG |
| EsaLHCI | GATC | Hin2I | C⇓CGG | Hpy85397P | CATG |
| Ese6II | CCWGG | Hin3I | CCSGG | Hpy85404P | CATG |
| Esp2I | CGWGG | Hin4II | CCTTC | Hpy85405P | CATG |
| Esp24I | CCWGG | Hin5I | CCGG | Hpy85406P | CATG |
| EspHK7I | CCWGG | Hin5II | GGNCC | Hpy85407P | CATG |
| EspHK22I | CCWGG | Hin6I | G⇓CGC | Hpy85408P | CATG |
| EspHK30I | CCWGG | Hin7I | GCGC | Hpy85409P | CATG |
| FaliI | CG⇓CG | Hin8II | CATG | Hpy99517P | GATC |
| FagI | GGGAC | Hin1056I | CGCG | Hpy788156P | TGCA |
| FatI | ⇓CATG | HinGUI | GCGC | Hpy788669P | TGGA |
| FauI | CCCGC | HinGUII | GGATG | Hpy790231P | ACNGT |
| FauBLI | CG⇓CG | HinP1I | G⇓CGC | Hpy790349P | CCTC |
| EbrI | GCINGC | HinS1I | GCGC | HpyAIP | CATG |
| FdiI | G⇓GWCC | HinS2I | GCGC | HpyAII | GAAGA |
| FgoI | C⇓TAG | Hinfi | G⇓ANTC | HpyAIII | GATG |
| FinI | GGGAC | HmaORFAP | CTAG | HpyAIV | GANTC |
| FinII | CCGG | HpaII | C⇓CGG | HpyAV | CCTTC |
| FinSI | GGCC | HphI | GGTGA | HpyAVIP | CCTC |
| FisI | GTAG | HpyIP | CATG | Hpy87AI | GANTC |
| FmuI | GGNC⇓C | HpyII | GAAGA | HpyA209P | CATG |
| FnuAI | G⇓ANTC | HpyIV | GANTC | HpyA214P | CATG |
| FnuAII | GATC | HpyV | TCGA | HpyA218P | CATG |
| FnuCI | ⇓GATC | HpyVIII | CCGG | HpyAORF263P | CCGG |
| FnuDI | GG⇓CC | Hpy8II | GTSAC | HpyAORF481P | ACNGT |
| FnuDII | CG⇓CG | Hpy26I | TGCA | HpyAORF483P | ACGT |
| FnuDIII | GCG⇓C | Hpy26II | TCGA | HpyAORF1537P | TGCA |
| FnuEI | ⇓GATC | Hpy51I | ⇓GTSAC | HpyAR250RFAP | CATG |
| Fnu4HI | GC⇓NGC | Hpy99I | CGWCG⇓ | HpyAR820RFAP | CATG |
| FokI | GGATG | Hpy99II | GTSAC | HpyAR840RFAP | CATG |
| Fsp16041 | CC⇓WGG | Hpy99III | GCGC | HpyBI | GT⇓AC |
| FspBI | C⇓TAG | Hpy99VIP | GATC | HpyCH4I | CATG⇓ |
| Fsp4HI | GC⇓NGC | Hpy99VIIIP | CCGG | HpyCH4II | CTNAG |
| HpyCH4III | ACN⇓GT | HpyF21II | GTAC | HpyF49II | GTSAC |
| HpyCH4IV | A⇓CGT | HpyF22I | ACNGT | HpyF49IV | GGGC |
| HpyCH4V | TG⇓CA | HpyF22II | CTNAG | HpyF49V | TGCA |
| HpyCR20RF1P | CCTC | HpyF23I | TCGA | HpyF50II | TCNGA |
| HpyCR20RF2P | CATG | HpyF24I | TCGA | HpyF51I | GTSAC |
| HpyCR20RF3P | GTSAC | HpyF24II | CTNAG | HpyF51II | ACNGT |
| HpyCR350RF1P | CATG | HpyF25I | CTNAG | HpyF52I | TCGA |
| HpyCR4RM1P | GTSAC | HpyF25II | GTSAC | HpyFS2II | CGCG |
| HpyCR9RM2P | GTSAC | HpyF26I | CGCG | HpyF52III | GTAC |
| HpyCR14RM2P | GTSAC | HpyF26II | GGGC | HpyF53I | GGCC |
| HR15RM1P | CATG | HpyF26III | TCGA | HpyF53H | GTAC |
| HpyCR29RM1P | CCTC | HpyF27I | CTNAG | HpyF54I | ACNGT |
| HpyCR29RM2P | GTSAC | HpyF27II | TCNGA | HpyF55I | ACNGT |
| HpyCR29RM3P | CATG | HpyF28I | TCNGA | HpyF55II | GANTC |
| HR35RM1P | CCTG | HpyF29I | GGCC | HpyF56I | ACNGT |
| HpyCR35RM2P | GTSAC | HpyF30I | TCGA | HpyF57I | GGCC |
| HpyCR38RM1P | CCTG | HpyF30II | GTNAG | HpyF58I | ACNGT |
| HpyCR38RM2P | GTSAG | HpyF31I | GTAC | HpyF59I | GTNAG |
| HpyCR38RM3P | CATG | HpyF31II | GTSAC | HpyF59II | GTAC |
| HpyF1I | GTSAC | HpyF32I | CTNAG | HpyF59III | TCGA |
| HpyF2II | GANTG | HpyF33I | TCNGA | HpyF60I | GANTC |
| HpyF3I | CTNAG | HpyF33II | GGCC | HpyF60II | CTNAG |
| HpyF4I | GTSAC | HpyF34I | CTNAG | HpyF61I | TCNGA |
| HpyF4II | CTNAG | HpyF34II | GTSAC HpyF61III | CGWGG | |
| HpyF5I | CTNAG | HpyF35I | TCGA | HpyF62I | ACNGT |
| HpyF5II | ACNGT | HpyF35II | ACGT | HpyF62II | TGGA |
| HpyF6I | GGATG HpyF35III | ACNGT | HpyF62III | GTSAC | |
| HpyF6II | GTSAC | HpyF35IV | GTSAC | HpyF63I | GGCC |
| HpyF6III | GTNAG | HpyF36I | GTSAC | HpyF64I | TCGA |
| HpyF7I | CTNAG | HpyF36II | GTAC | HpyF64II | ACNGT |
| HpyF9I | GTSAC HpyF36III | TGCA | HpyF64III | TCNGA | |
| HpyF9II | CTNAG | HpyF37I | CTNAG | HpyF64IV | CGCG |
| HpyF9III | ACNGT | HpyF38I | GANTG | HpyF64V | CTNAG |
| HpyF10I | GCGC | HpyF38II | TGCA | HpyF65I | ACNGT |
| HpyF10II | GANTC | HpyF40I | ACNGT | HpyF65II | TCGA |
| HpyF10IV | GTAC | HpyF40II | TCGA | HpyF65III | GTAC |
| HpyF10V | GGCC | HpyF40III | GTSAC | HpyF66I | GGNCC |
| HpyF11I | CTNAG | HpyF4II | ACNGT | HpyF66II | CTNAG |
| HpyF11II | TCNGA | HpyF4III | CTNAG | HpyF66III | GTAC |
| HpyF12I | ACNGT | HpyF42I | GGCC | HpyF66IV | TCGA |
| HpyF12II | TCNGA | HpyF42II | ACNGT | HpyF67I | CTNAG |
| HpyF13I | GTSAC | HpyF42III | TCNGA | HpyF67II | TGCA |
| HpyF13II | CTNAG | HpyF42IV | TCGA | HpyF67III | GGATG |
| HpyF13III | AGGT | HpyF43I | CCGG | HpyF68I | ACNGT |
| HpyF13IV | GTAC | HpyF44I | GANTC | HpyF68II | CTNAG |
| HpyF14I | CGCG | HpyF44III | TG⇓CA | HpyF69I | ACNGT |
| HpyF14III | TCGA | HpyF44V | GTAC | HpyF69II | GGCC |
| HpyF15I | CGCG | HpyF45I | TCGA | HpyF70I | CTNAG |
| HpyF15II | TCNGA | HpyF45II | TGCA | HpyF71I | TCGA |
| HpyF16I | TCGA | HpyF46I | ACNGT | HpyF71II | GGNCC |
| HpyF17I | TCNGA | HpyF46IV | TCNGA | HpyF71III | GANTC |
| HpyF18I | GANTG | HpyF46V | GGCC | HpyF72I | GGCC |
| HpyF19I | CTNAG | HpyF48I | GTSAC | HpyF72II | CTNAG |
| HpyF19II | TCNGA | HpyF48II | ACNGT | HpyF72III | GANTC |
| HpyF20I | ACNGT | HpyF48III | TGCA | HpyF73II | TCGA |
| HpyF21I | CTNAG | HpyF49I | TCGA | HpyF73III | GGCC |
| HpyF73IV | GGNGG | Lla497I | CCWGG | MthFI | CTAG |
| HpyF74I | ACNGT | LlaAI | ⇓GATC | MthTI | GGCC |
| HpyF74II | ACGT | LlaDII | GCNGC | MthZI | C⇓TAG |
| HpyHPK5I | CTNAG | LlaDCHI | GATC | MvaI | CC⇓WGG |
| HpyHPK5II | GATC | LlaKR2I | GATC | MvaAI | CGCG |
| HpyIn18AP | CATG | LlaMI | CCNGG | MvnI | CG⇓CG |
| HpyIn34AP | CATG | Lsp1109I | GCAGC | NanII | GATC |
| HpyIn44AP | CATG | Lsp1109II | GATC | NcaI | GANTC |
| HpyIn227P | CATG | LweI | GCATC | NciI | CC⇓SGG |
| HpyJ101P | CATG | MaeI | C⇓TAG | NciAI | GATC |
| HpyJF13P | CATG | MaeII | A⇓CGT | NcuI | GAAGA |
| HpyJF15P | CATG | MaeIII | ⇓GTNAC | NdeII | ⇓GATC |
| HpyJF16P | CATG | MaeK81II | G⇓GNCC | NflI | GATC |
| HpyJF36P | CATG | MarI | AGCT | NflAII | GATC |
| HpyJF37P | CATG | MboI | ⇓GATC | NflBI | GATC |
| HpyTh38P | CATG | MboII | GAAGA | NgoAII | GGCC |
| HpyJF43P | CATG | MchAII | GG⇓CC | NgoAVIP | GATC |
| HpyJF70P | CATG | MeuI | GATC | NgoAVIIP | GCSGC |
| HpyJF72P | CATG | MfoI | GGWCC | NgoAORFC7I7P | GGTGA |
| HpyJF73P | CATG | MfoAI | GG⇓CC | NgoBIIP | GGCC |
| HpyJF79P | CATG | Mg114481I | CC⇓SGG | NgoBVIII | GGTGA |
| HpyJF82P | CATG | MgoI | GATC | NgoCII | GGCC |
| HpyJF83P | CATG | MjaI | CTAG | NgoDVIII | GGTGA |
| HpyJF84P | CATG | MjaII | GGNCC | NgoDXIV | GATC |
| HpyJP26I | TGCA | MjaIII | GATC | NgoEII | GCGC |
| HpyJP26II | TCGA | MjaV | GTAC | NgoFVII | GCSGC |
| HpyNI | CCNGG | MkrAI | ⇓GATC | NgoJVIII | GGTGA |
| HpyOK99P | CATG | MliI | GGWCC | NgoLIIP | GGCC |
| HpyOK102P | CATG | MltI | AG⇓CT | NgoMIIP | GGCG |
| HpyOK104P | CATG | Mlu2300I | CCWGG | NgoMVIII | GGTGA |
| HpyOK106P | CATG | MluCI | AATT | NgoNII | GGCC |
| HpyOK107P | CATG | MlyI | GAGTC | NgoPII | GG⇓CC |
| HpyOK108P | CATG | MmeII | GATC | NgoSII | GGCC |
| HpyOK111P | CATG | MniI | GGCC | NgoTII | GGCC |
| HpyOK113P | GATG | MnilI | CCGG | NlaI | GGCC |
| Hpy0K115P | CATG | MnII | CCTC | NlaII | ⇓GATC |
| Hpy0K129P | CATG | MnnII | GGCC | NlaIII | CATG⇓ |
| Hpy0K134P | CATG | MnnIV | GCGC | NIaX | CCNGG |
| Hpy99ORJF433P | ACNGT | MnoI | C⇓CGG | NlaDI | GATC |
| HsoI | G⇓CGC | MnoIII | GATC | NlaDII | GGNCC |
| Hsp2I | GGWCC | MosI | GATC | NliII | GGWCC |
| Hsp92II | CATG⇓ | MphI | CCWGG | Nli3877II | GGWCC |
| HspAI | G⇓CGC | Mph1103II | GATC | NmeAI | GATC |
| ItaI | GC⇓NGC | MseI | TITAA | NmeAORF1500P | CCWGG |
| Kox165I | CCWGG | MspI | G⇓CGG | NmeBI | GACGC |
| Kpn10I | CCWGG | Msp24I | GGNCC | NmeB1940P | GATC |
| Kpn13I | CCWGG | Msp67I | CC⇓NGG | NmeBL2P | GATC |
| Kpn14I | GGWGG | Msp67II | GATC | NmeBL859I | GATC |
| Kpn16I | CCWGG | Msp199I | CCGG | NmeBL915P | GATC |
| Kpn2kI | ⇓CCNGG | MspAI | GGWCC | NmeBORF1290P | CCWGG |
| Kpn49kII | ⇓CCSGG | MspBI | GATC | NmeBORF1896P | GATC |
| KspHK12I | CCWGG | MspR9I | CCINGG | NmeBS847P | GATC |
| KspHK14I | CCWGG | MthI | GATC | NmeCI | ⇓GATC |
| Kzo9I | ⇓GATC | Mth1047I | GATC | NmeNL4627P | GATC |
| Kzo491 | G⇓GWCG | MthAI | GATC | NmuAII | GGWCC |
| LfeI | GGAGG | MthBI | GGNCC | NmuCI | ⇓GTSAC |
| NmuDI | GATC | PspGI | ⇓GCWGG | SecII | CCGG |
| NmuEI | GATC | PspPI | G⇓GNCC | SelI | ⇓CGCG |
| NmuEII | GGNCC | Ral8I | GGATC | SelAI | GGNCC |
| NmuSI | GGNCC | RalF40I | ⇓GATC | SenPI | CCNGG |
| NovII | GANTC | Rlu1I | GATC | SeqORFC272P | GGATG |
| NphI | ⇓GATC | RmaI | C⇓TAG | SfaI | GG⇓CC |
| NsiAI | GATC | Rma485I | CTAG | SfaGUI | CCGG |
| NsiHI | GANTC | Rma486I | CTAG | SfaNI | GCATC |
| NspIV | G⇓GNCC | Rnia49OI | CTAG | SflHK17941 | CCWGG |
| Nsp7l2lI | G⇓GNCC | Rma495I | CTAG | SflHK2374I | CCWGG |
| NspAI | GATC | Rma496I | CTAG | SflHK2731I | CCWGG |
| NspDII | GGWGC | Rma497I | CTAG | SflHK6873I | CCWGG |
| NspGI | GGWCC | Rma500I | CTAG | SflHK7234I | CCWGG |
| NspHII | GGWCC | Rma5OlI | CTAG | SflHK7462I | CCWGG |
| NspKI | GGWCC | Rma5O3I | CTAG | SflHK8401I | CGWGG |
| NspLII | GGNCC | Rma5O6I | CTAG | SflHK10695I | CCSGG |
| NspLKI | GG⇓GG | Rma5O9I | CTAG | SflHK10790I | CCWGG |
| NsuI | GATC | Rma510I | CTAG | SflHK11086I | CGSGG |
| NsuDI | GATC | Rnia515I | CTAG | SflHK10871I | CCSGG |
| OchI | GGCC | Rrna516I | CTAG | SflHK11572I | CCSGG |
| OihORF3333P | GCNGC | Rma5l7I | CTAG | SflHK115731I | CCSGG |
| OtuI | AGCT | Rma518I | CTAG | Sfl2aI | CGWGG |
| OtuNI | AGCT | Rma519I | CTAG | Sfl2bI | CCWGG |
| OxaI | AGCT | Rma522I | CTAG | SfnI | GGWCC |
| Pae181I | CCSGG | RsaI | GT⇓AC | Sgh1835I | GGWCC |
| PaeIMORF3201P | GCWGC | RshII | CCSGG | Sgr20I | CCWGG |
| PaiI | GGCC | SagI | GGCC | ShaI | GGGTG |
| PalI | GG⇓CC | SaiI | GGGTC | SimI | GGGTC |
| Pde12I | G⇓GNCC | SalAI | GATC | SinI | G⇓GWCC |
| Pde133I | GG⇓CC | SaiHI | GATC | SinAI | GGWCC |
| Pde137I | G⇓CGG | SatI | GC⇓NGC | SinBI | GGWCC |
| Pei9403I | GATC | Sau2I | GGNCC | SinCI | GGWCC |
| PfaI | GATC | SauSI | GGNCC | SinDI | GGWCC |
| PfeI | G⇓AWTC | Saul3I | GGNCC | SinEI | GGWCC |
| Pfl19I | GGWCC | Saul4I | GGNCC | SinFI | GGWCC |
| PflAI | CGCG | Sau15I | GATC | SinGI | GGWCC |
| PflKI | GG⇓CC | Saul6I | CCWGG | SinHI | GGWCC |
| PhaI | GCATC | Sau17I | GGNCC | SinJI | GGWCC |
| PhoI | GG⇓CC | 5au96I | G⇓GNCC | SinMI | GATC |
| PlaI | GG⇓CC | 5au5571 | GGNCC | SleI | ⇓CCWGG |
| PlaAII | GT⇓AC | 5au6782I | GATC | SmiMBI | GATG |
| PleI | GAGTC | Sau3AI | ⇓GATC | SmuI | CCGGC |
| Ple214I | GGCG | SauBI | GGNCC | SmuEI | G⇓GWGG |
| Pme35I | CCGG | SauCI | GATC | SmuUORF504P | GATC |
| PolI | GGWCC | SauDI | GATC | SniI | CC⇓WGG |
| PpaAII | T⇓CGA | SauEI | GATC | SplIII | GGCG |
| Pph288I | GATC | SauFI | GATC | Spn19FORF24P | GATC |
| Pph1579I | GGNGC | SauGI | GATC | SpnHGORF3P | GATC |
| Pph1773I | GGNCC | SauMI | ⇓GATC | SpnORF1850P | GATC |
| PpsI | GAGTC | SbvI | GG⇓CC | SpnRORF1665P | GATC |
| PpuI | GGCG | SceAI | CGCG | SscL1I | G⇓ANTC |
| PseI | GGNCC | Scg2I | GCWGG | Sse9I | ⇓AATT |
| PspI | GGNCC | SchI | GAGTC | SsiI | CCGC |
| Psp03I | GGWC⇓C | SciNI | G⇓CGC | SsiAI | ⇓GATC |
| Psp6I | CCWGG | ScrFI | GC⇓NGG | SsiBI | ⇓GATC |
| Psp29I | GGCC | SdyI | GGNCC | Ss1I | CC⇓WGG |
| SsoII | ⇓CCNGG | Tru1I | T⇓TAA | Uba61I | GGCC |
| Ssp2I | CCSGG | Tru9I | T⇓TAA | Uba62I | GGWGC |
| SspAI | ⇓CCWGG | Tru28I | GGWCC | Uba81I | CCWGG |
| SspD5I | GGTGA | TscI | ACGT⇓ | Uba82I | CCWGG |
| Ssu211I | GATC | Tsc4aI | TCGA | Uba1097I | GGCC |
| Ssu212I | GATC | TseI | G⇓CWGC | Uba1099I | GGNCC |
| Ssu220I | GATC | TseBI | GGWGC | Uba1101I | GATC |
| R1.Ssu2479I | GATG | TseCI | AATT | Uba1114I | CCWGG |
| R2.Ssu2479I | GATC | Tsp1I | ACTGG | Uba1118I | CCWGG |
| R1.Ssu4109I | GATG | Tsp32I | T⇓CGA | Uba1120I | CCWGG |
| R2.Ssu4109I | GATC | Tsp32II | T⇓CGA | Uba1121I | CCWGG |
| R1.Ssu4961I | GATC | Tsp45I | ⇓GTSAC | Uba1125I | CCWGG |
| R2.Ssu4961I | GATC | Tsp49I | ACGT⇓ | Uba1128I | CCGG |
| R1.Ssu8074I | GATC | Tsp132I | GGCC | Uba1131I | GGWCC |
| R2.Ssu80741 | GATC | Tsp133I | GATC | Uba1134I | GGNCC |
| R1.Ssu11318I | GATC | Tsp266I | GGCC | Uba1140I | GGCC |
| R2.Ssu11318I | GATC | Tsp273II | GGCC | Uba114II | CCGG |
| R1.SsuDAT1I | GATC | Tsp281I | GGCC | Uba1146I | GGCC |
| R2.SsuDAT1I | GATC | Tsp301I | GGWCC | Uba1147I | GGCC |
| SsuRBI | GATC | Tsp358I | TCGA | Uba1150I | GGCC |
| Sth117I | CC⇓WGG | Tsp505I | TCGA | Uba1152I | GGCC |
| Sth132I | CCCG | Tsp509I | ⇓AATT | Uba1153I | GGCC |
| Sth134I | C⇓CGG | Tsp510I | TCGA | Uba1155I | GGCC |
| Sth368I | ⇓GATC | Tsp560I | GGCC | Uba1160I | GGNCC |
| Sth455I | CCWGG | TspAI | CCWGG | Uba1164I | GGNCC |
| SthSt0IP | GCNGC | TspAK13D21I | TCGA | Uba1169I | GGCC |
| SthSt8IP | GATC | TspAK16D24I | TCGA | Uba1171I | CCWGG |
| StsI | GGATG | Tsp4CI | ACN⇓GT | Uba1174I | GGCC |
| StyD4I | ⇓CCNGG | TspDTI | ATGAA | Uba1175I | GGCC |
| SuaI | GGICC | TspEI | ⇓AATT | Uba1176I | GGCC |
| SulI | GGCC | TspGWI | ACGGA | Uba1177I | GATC |
| SynI | GGWCC | TspIDSI | ACGT | Uba1178I | GGCC |
| TaaI | ACN⇓GT | TspNI | TCGA | Uba1179I | GGCC |
| Tail | ACGT⇓ | TspVi4AI | TCGA | Uba1181I | CCWGG |
| TaqI | T⇓GGA | TspVil3I | TCGA | Uba1182I | GATC |
| Taq20I | TCGA | TspWAM8AI | ACGT | Uba1183I | GATC |
| Taq52I | G⇓CWGC | TspZNI | GGCC | Uba1185I | CCWGG |
| TaqXI | CC⇓WGG | TteAI | GGCC | Uba1189I | CCWGG |
| TasI | ⇓AATT | Tth24I | TCGA | Uba1193I | CCWGG |
| TauI | GCSG⇓C | TtbHB8I | T⇓GGA | Uba1204I | GATC |
| Tbr51I | TCGA | TthRQI | TCGA | Uba1207I | GGCC |
| TceI | GAAGA | TtmI | ACGT | Uba1208I | GGCC |
| TdeI | GATC | TtnI | GGCG | Uba1209I | GGCC |
| TdeIII | GGNCC | TvoORF1413P | CGSGG | Uba1210I | GGGC |
| TerORFS1P | GATG | TvoORF1416P | CCWGG | Uba1214I | GGGC |
| TerORIFSI8P | GCSGC | Uba4I | GATC | Uba1218I | CCWGG |
| TfiI | G⇓AWTC | Uba9I | GGCC | Uba1223I | GGCC |
| TfiA3I | TCGA | Uba11I | CCWGG | Uba1228I | GGCC |
| TfiTok4A2I | TCGA | Uba13I | CCWGG | Uba1230I | GGCC |
| TfiTok6A1I | TCGA | Uba17I | CCNGG | Uba1231I | GGCC |
| TflI | TCGA | Uba20I | CCWGG | Uba1235I | GGCC |
| ThaI | CG⇓CG | Uba41I | CCSGG | Uba1243I | CCWGG |
| TmaI | CGCG | Uba42I | CCSGG | Uba1249I | GGWCG |
| Tmu1I | GCSGG | Uba48I | GGWCC | Uba1259I | GATC |
| TruI | GGWCG | Uba54I | GGCC | Uba1267I | CGGG |
| TruII | GATC | Uba59I | GATC | Uba1272I | GGWCC |
| Uba1278I | GGWCC | VchO85I | GGNCC | Uba1372I | CCSGG |
| Uba1280I | GCSGG | VchO90I | GGNCC | Uba1373I | GGWCC |
| Uba1288I | GGCC | VhaI | GGCC | Uba1376I | CCSGG |
| Uba1292I | GGCC | Vha44I | GATG | Uba1377I | GGCC |
| Uba1293I | GGCC | Vha1168I | GGCC | Uba1378I | CCSGG |
| Uba1304I | GGWCC | VniI | GGCC | Uba1388I | GGCC |
| Uba1314I | GGWCC | VpaK11I | GGWCC | Uba1389I | CCSGG |
| Uba1317I | GATC | VpaK15I | GGNCC | Uba1391I | CCNGG |
| Uba1318I | CCSGG | VpaK25I | GGNCG | Uba1392I | GGCC |
| Uba1319I | GGCC | VpaK65I | GGWCC | Uba1395I | GGCC |
| Uba1321I | CGCG | VpaK7AI | GGWCC | Uba1401I | CCSGG |
| Uba1322I | GGCC | VpaK9AI | GGNCC | Uba1404I | CGCG |
| Uba1323I | GATC | VpaK11AI | ⇓GGWCC | Uba1405I | CGCG |
| Uba1336I | GGCC | VpaK13AI | GGWCC | Uba1408I | GGCC |
| Uba1338I | CCGG | VpaK19AI | GGNCC | Uba1410I | CGWGG |
| Uba1347I | CCSGG | VpaK19BI | GGNCC | Uba1413I | GGWCC |
| Uba1355I | CCGG | VpaK11CI | GGWCC | Uba1418I | GGCC |
| Uba1366I | GATC | VpaK11DI | GGWCC | Uba1422I | GGCC |
| Uba1370I | GCSGG | VpaKutAI | GGNCC | Uba1423I | CCSGG |
| Uba1424I | CCSGG | | | | |
| Uba1428I | CCWGG | | | | |
| Uba1429I | GGCC | | | | |
| Uba1433I | AGCT | | | | |
| Uba1438I | GGWCC | | | | |
| Uba1439I | CCGG | | | | |
| Uba1441I | AGCT | | | | |
| Uba14461 | CGCG | | | | |
| Uba14491 | GGCC | | | | |
| Uba14501 | GGCC | | | | |
| UnbI | ⇓GGNGC | | | | |
| Uth549I | GGCC | | | | |
| Uth554I | GGWCG | | | | |
| Uth555I | GGCC | | | | |
| Uth557I | GGCC | | | | |
| Uur960I | GC⇓NGG | | | | |
| Van911II | GGCC | | | | |
| VchO66I | GGNCC | | | | |
| VpaKutBI | GGNCG | | | | |
| VpaKutJI | GGNCG | | | | |
| XspI | C⇓TAG | | | | |
| ZanI | CC⇓WGG | | | | |
| VpaKutBI | GGNCC | | | | |
| VpaKutJI | GGNCC | | | | |
| XspI | C⇓TAG | | | | |
| ZanI | CC⇓WGG | | | | |

1. A method of identifying an integrant integration site, comprising:
(a) obtaining a nucleic acid molecule comprising at least one integrant at an integration site and at least one first restriction site (N1 site) cleavable by a first restriction enzyme (N1), wherein the integrant comprises in the following order:
(i) a first terminal repeat, comprising a target end and a terminal repeat-specific primer (TRP) binding site, which can stably bind a TRP,
(ii) at least one second restriction site (N2 site) cleavable by a second restriction enzyme (N2), and
(iii) a second terminal repeat, comprising a non-target end and a sequence, which can stably bind a TRP, and which is in the same orientation as the TRP binding site in the first terminal repeat,
wherein there are no N1 sites or N2 sites in the TRP binding site or between the target end and the TRP binding site, and wherein there are no N1 sites between the N2 site closest to the non-target end and the non-target end;
(b) digesting the nucleic acid molecule with N1 and N2 to yield a population of nucleic acid fragments, wherein at least some of the fragments have at least one N1 end;
(c) ligating an extension-dependent linker to at least some of the N1 ends to produce a population of linkered fragments;
(d) contacting the linkered fragments with the TRP;
(e) extending the TRP to yield at least one extension product having a linker-specific primer (LSP) binding site complementary to a LSP;
(f) amplifying the linkered fragments and extension product(s) with TRPs and LSPs to yield at least one amplification product; and
(g) sequencing at least one amplification product to yield at least one nucleic acid sequence flanking the target end, thereby identifying at least one integrant integration site.

2. The method of claim 1, wherein the integrant is a virus, a transposon, or an integrating gene therapy vector.

3. The method of claim 2, wherein the integrant is a virus.

4. The method of claim 3, wherein the integrant is murine leukemia virus (MLV) or human immunodeficiency virus 1 (HIV-1).

5. The method of claim 1, wherein the TRP binding site is no more than about 200 base pairs from the target end.

6. The method of claim 1, wherein the target end is the 3′ end of the integrant.

7. The method of claim 1, wherein the target end is the 5′ end of the integrant.

8. The method of claim 1, wherein the nucleic acid molecule is genomic DNA.

9. The method of claim 8, wherein the nucleic acid molecule is human genomic DNA.

10. The method of claim 1, wherein N1 is no more than a 5-base cutter.

11. The method of claim 10, wherein N1 is no more than a 4-base cutter.

12. The method of claim 1, wherein N2 cuts the nucleic acid molecule less frequently than does N1.

13. The method of claim 11, wherein N1 is MseI, RsaI, TaqI, Tri1I or RsaI.

14. The method of claim 1, wherein N2 is PstI or EcoRI.

15. The method of claim 1, wherein the population of nucleic acid fragments comprise an average length of no more than about 300 base pairs.

16. The method of claim 15, wherein the average fragment length is no more than about 100 base pairs.

17. The method of claim 1, wherein the nucleic acid molecule is co-digested with N1 and N2.

18. The method of claim 17, wherein N1 and N2 produce incompatible ends.

19. The method of claim 1, wherein the nucleic acid molecule is sequentially digested with N1 and N2.

20. The method of claim 19, wherein N1 and N2 produce compatible ends.

21. The method of claim 19, wherein the nucleic acid molecule is first digested with N1 and then digested with N2.

22. The method of claim 21 further comprising isolating linkered fragments prior to digesting with N2.

23. The method of claim 1, wherein the integrant further comprises at least one N1 site.

24. The method of claim 1, wherein the method is performed in no more than 14 days.

25. The method of claim 1, wherein the method is performed in no more than 7 days.

26. The method of claim 1, wherein the nucleic acid sequence flanking the target end is no more than about 75 base pairs.

27. The method of claim 26, wherein the nucleic acid sequence flanking the target end is no more than about 30 base pairs.

28. The method of claim 1, wherein at least 200 integration sites are identified.

29. The method of claim 28, wherein at least 500 integration sites are identified.

30. A method of determining the risk potential of an integrating gene therapy vector, comprising:
isolating a nucleic acid molecule, comprising at least one integrated integrating gene therapy vector and at least one reference point, from a treated cell
identifying integration sites of the gene therapy vector according to the method of claim 1; and
mapping integration sites in relation to at least one reference point;
wherein the map of integration sites provides information about the risk potential of the integrating gene therapy vector.

31. The method of claim 30, wherein the treated cells comprise mammalian cells.

32. The method of claim 31, wherein the mammalian cells comprise human cells.

33. The method of claim 32, wherein the human cells are isolated from a subject to whom the treated cells are to be administered.

34. The method of claim 32, wherein the human cells are isolated from a subject to whom the treated cells were administered.

35. The method of claim 34, wherein the treated cells were administered to the subject as a medical treatment.

36. The method of claim 30, wherein the nucleic acid molecule comprises genomic DNA.

37. The method of claim 30, wherein the integrating gene therapy vector comprises all or part of the genome from MLV or HIV-1.

38. The method of claim 36, wherein the reference point comprises actively transcribed regions of the nucleic acid molecule; or telomeres.

39. The method of claim 38, wherein reference points in actively transcribed regions comprise translation start sites, transcription start sites, midpoints of coding regions, or stop codons.

40. The method of claim 39, wherein the risk potential of the integrating gene therapy vector is relatively high when substantial numbers of integration sites are located near actively transcribed regions of the nucleic acid molecule.

41. The method of claim 39, wherein the risk potential of the integrating gene therapy vector is relatively low when the distribution of integration sites is substantially random in relation to actively transcribed regions of the nucleic acid molecule.

42. The method of claim 30, wherein at least 500 integration sites are mapped.

43. The method of claim 42, wherein at least 750 integration sites are mapped.

44. The method of claim 43, wherein substantially all integration sites are mapped.


SEQUENCE LISTING (14 sequences)

SEQ ID NO: 1 - DNA, 36 bp, Artificial Sequence
  Linker Plus Strand
gtaatacgactcactatagggctccgcttaagggac

SEQ ID NO: 2 - DNA, 17 bp, Artificial Sequence
  Linker Minus Strand
tagtcccttaagcggan

SEQ ID NO: 3 - DNA, 22 bp, Artificial Sequence
  Primer
gtaatacgactcactatagggc

SEQ ID NO: 4 - DNA, 19 bp, Artificial Sequence
  Primer
agggctccgcttaagggac

SEQ ID NO: 5 - DNA, 25 bp, Artificial Sequence
  Primer
gacttgtggtctcgctgttccttgg

SEQ ID NO: 6 - DNA, 25 bp, Artificial Sequence
  Primer
ggtctcctctgagtgattgactacc

SEQ ID NO: 7 - DNA, 22 bp, Artificial Sequence
  Primer
agtgcttcaagtagtgtgtgcc

SEQ ID NO: 8 - DNA, 23 bp, Artificial Sequence
  Primer
gtctgttgtgtgactctggtaac

SEQ ID NO: 9 - DNA, 38 bp, Artificial Sequence
  RsaI-compatible linker plus strand
gtaatacgactcactatagggcacgcgtggtccatggg

SEQ ID NO: 10 - DNA, 12 bp, Artificial Sequence
  Rsa-compatible linker minus strand
cccatggaccan

SEQ ID NO: 11 - DNA, 22 bp, Artificial Sequence
  RsaI-compatible linker primer
gtaatacgactcactatagggc

SEQ ID NO: 12 - DNA, 19 bp, Artificial Sequence
  RsaI-compatible linker nested primer
actatagggcacgcgtggt

SEQ ID NO: 13 - DNA, 21 bp, Artificial Sequence
  MLV 5′ LTR primer
tagcttgccaaacctacaggt

SEQ ID NO: 14 - DNA, 21 bp, Artificial Sequence
  MLV 5′ LTR primer
acctacaggtggggtctttca